(19) United States
(12) Patent Application Publication
(10) Pub. No.: US 2004/0266732 A1
(43) Pub. Date: Dec. 30, 2004

(54) Therapeutic agents, methods, and treatments

(76) Inventors: Jorge Galvez, Valencia (ES); Javier Llompart, Valencia (ES); Kollol Pal, Needham, MA (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/836,638

(22) Filed: Apr. 30, 2004

Related U.S. Application Data
(63) Continuation-in-part of application No. 10/251,616, filed on Sep. 20, 2002.
(60) Provisional application No. 60/556,317, filed on Mar. 25, 2004.

(51) Int. Cl. A61K 31/675; A61K 31/66; A61K 31/454; A61K 31/445

(52) U.S. Cl. 514/79; 514/114; 514/319; 514/408

(57) ABSTRACT

Compositions and uses associated with the MT103 family of compounds are disclosed. Particular structural features and properties of the compounds are described in detail. Uses include administering an MT103 family member to a patient for therapeutic purposes. Compositions include chemicals belonging to the MT103 family and pharmaceuticals that contain such chemicals. Methods of treating cells are also described.

### CROSS REFERENCE TO RELATED APPLICATIONS

This application claims priority to U.S. patent Ser. No. 60/556,317 filed March 25, 2004, entitled “THERAPEUTIC AGENTS AND CORRESPONDING TREATMENTS”, and is a continuation-in-part to U.S patent application Ser. No. 10/251,616 filed September 20, 2002, entitled “THERAPEUTIC AGENTS AND CORRESPONDING TREATMENTS”, which are hereby incorporated by reference herein.

### FIELD OF THE INVENTION

The application is generally related to methods of treating patients with chemical agents, and methods of inhibiting cell growth, including cancer.

### BACKGROUND OF THE INVENTION

Cancer is a disease that afflicts many people and is a leading cause of death in humans and non-human animals. Cancers typically involve cells that grow by uncontrolled growth of the cells that creates many new cells. Many anti-cancer drugs are agents that inhibit or stop cell growth.

Many anti-cancer drugs are known to be effective against cancers and tumor cells, but some cancers and tumors respond poorly to these drugs. Further, many anti-cancer drugs also destroy other cells in the body. Thus, new anti-cancer drugs are desired, and drugs that are able to target specific cancer types can provide useful therapeutic options.

Agents that inhibit cell growth are useful as anti-cancer drugs. The National Cancer Institute (NCI) is an agency of the United States government that is involved in the testing of anti-cancer drugs. NCI often conducts initial screening tests of potential anti-cancer drugs using a three cell line test. Each of the three cell lines is a type of cancerous cell. The cells are exposed to the drug candidates, and the drugs' effectiveness in stopping cell growth and/or killing the cells is measured.

The NCI typically tests the most promising drugs with a further battery of approximately 60 cell lines, and the dose of the drug that is required to stop cell growth and to kill cells is measured. The dose of the drug that is required to inhibit approximately 50% of the growth of a cancer cell is reported as the GI₅₀ concentration of the drug. The lower the GI₅₀, the more effective is the anti-cancer drug. The G150 is sometimes reported in the units of −log (GI₅₀), so that the higher the value for −log (GI₅₀), the more effective is the anti-cancer drug. The dose of the drug that is required to stop approximately 100% of cell growth is reported as the total growth inhibition (TGI) concentration of the drug. The dose of the drug that is required to reduce the number of the cells to 50% of the original number of cells is referred to as the LC₅₀ concentration. The lower the TGI or LC₅₀, the more potent is the anti-cancer drug.

### SUMMARY OF THE INVENTION

The invention includes embodiments related to the MT103 family of therapeutic compounds, as shown for example, in Formulas 1(a) and 1(b), below. An embodiment of the invention is a method of using an MT103 family member for treatment of patients, for example, as a cancer therapeutic, antibacterial, antifungal, apoptosis agent, protein kinase agent, and/or hormonal antagonist. Another embodiment is using a MT103 family member as a therapeutic, antibacterial, antifungal, apoptosis agent, protein kinase agent, and /or hormonal antagonist. Another embodiment is a therapeutic, antibacterial, antifingal, apoptosis agent, protein kinase agent, and /or hormonal antagonist that comprises an MT103 family member, e.g., a chemical according to one of Formulas 1-70 below. Embodiments of the invention include compositions and methods for treating a patient, including providing to, or administering to, a patient a therapeutically effective amount of a composition comprising a chemical as in Formulas 1-70.

Another embodiment is a chemical according to one of Formulas 1-70 below, or a species thereof. Another is embodiment is a pharmaceutical composition associated with a chemical according to one of Formulas 1-70 below, or a species thereof. Another embodiment is a method that includes exposing a cell to a composition comprising a chemical according to one of Formulas 1-70 below, or a species thereof, e.g., for diagnosis, testing, screening, or treatment in vitro or in vivo.

### BRIEF DESCRIPTION OF THE FIGURES

FIG. 1 depicts GI₅₀ values for MT103 for a variety of cell lines, with the GI₅₀ values being plotted as −logGI₅₀; and

FIG. 2 depicts the inhibition of the NCI-H226 human non-small cell lung cancer cell line by MT103.

FIG. 3 is a graph showing inhibition of tumor growth in vivo using a member of the MT103 family, as described in greater detail in Example 7.

FIG. 4 is a graph of test results showing that a member of the MT103 family to be non-toxic, as described in greater detail in Example 7.

FIG. 5 is a graph of test results showing a member of the MT103 family was more effective than the anti-cancer drug cisplatin for slowing tumor growth in vivo, as described in greater detail in Example 8.

FIG. 6 shows dose response curves for MT103 administered at therapeutically suitable amounts, as demonstrated with three lung cancer cell line, as described in greater detail in Example 5.

### DETAILED DESCRIPTION OF THE PREFERRED EMBODIMENTS

An anti-cancer agent referred to herein as MT103 is disclosed (Formula 2), along with derivatives of this molecule that are expected to have an anti-cancer activity. These compounds were developed using computer models that analyze topological features of molecules and help to predict which ones will be effective. The predictive power of these processes have been verified by successful in vitro and in vivo tests of candidate compounds, including those set forth in the Examples. Variations of the MT103 molecule are described herein that have structural similarity to MT103 that is expected to give them anti-cancer properties.

The creation of new anti-cancer drugs is a challenging process. An important step is the selection of drug candidates for initial screening. Many approaches for selecting these drug candidates are used. One approach is to use computer modeling to design molecules that have physicochemical properties that are useful as anti-cancer agents.

Computer Modeling

A topological computer modeling program that incorporates a molecular shape learning system has been used to identify the new family of drugs exemplified by MT103. The modeling program takes topological information about chemicals that are known to be effective anti-cancer drugs, and in a next step identifies common topological features that the drugs should share to show activity in the property under study. Then the program identifies new chemicals that have the common topological features. The program is designed not only to identify chemicals that are anti-cancer compounds but also to identify chemicals that are useful to combat specific types of cancer. MT103 was identified by the program as a chemical that would inhibit the growth of cancer cells. Further, MT103 was identified as a compound having particular efficacy against non-small lung cancer cells. The fact that a compound was successfully identified with that function is proof of the efficacy and utility of the compounds predicted by the computer model.

The computer modeling approach relies on molecular topology to determine physicochemical properties of molecules. The topological approach relies on mathematical means to describe and construct descriptive computer models. Through these models, it is possible to forward engineer specific structural activity relations of a molecular charge density alone or in response to adjacent electrotopological features. The topological approach accounts for the true structural invariant of a molecule that is not affected by vibrational or conformational changes. Aspects of this approach are set forth by Galvez in J. Gálvez et al., J. Chem Inf. Comput. Sci., Vol. 34, No. 3, 1994; J. Gálvez et al., J. Chem Inf. Comput. Sci., Vol. 34, No. 5, 1994; J. Gálvez et al., J. Chem Inf. Comput. Sci., Vol. 35, No. 2, 1995; J. Gálvez et al., Bioorganic & Medicinal Chemistry Letters, Vol. 6, No. 19, 1996; J. Gálvez et al., Journal of Molecular Graphics, Vol. 14, 1996; J. Gálvez, Journal of Molecular Structure (Theochem), Vol. 429, 1998; J. V. de Julián-Ortiz, Journal of Molecular Graphics and Modeling, Vol. 16, 1998; Jesus V. de Julián-Ortiz et al., Journal of Medicinal Chemistry, Vol. 42, No. 17; Rafael Gozalbes et al., Antimicrobial Agents and Chemotherapy, Vol. 44, No. 10, October 2000; M. J. Duart et al., Journal of Computer-Aided Molecular Design, Vol. 15, 2001; L. Lahuerta Zamora et al., Analytical Chemistry, Vol. 73, No. 17, Sep. 1, 2001.

Trained models predict the bioactive topology of molecules and can be readily interpreted to guide the design of new active compounds. This approach combines three advances: a representation that characterizes surface shape such that structurally diverse molecules exhibiting similar surface characteristics are treated as similar; a new machine learning methodology that can accept multiple orientations and conformations of both active and inactive molecules; and an iterative process that applies intermediate models to generate new molecular orientations to produce better predictive models. Two aspects of the program described above, the method of iterative reposing objects to produce better models and the method of training a model when each object has multiple representations, are applicable not only to biological activity modeling but also to other physicochemical characteristics.

The efficacy of the compounds generated by the topological computer modeling program can be confirmed using routine screening by using known cancer cell lines. Cell lines are available from NCI, American Tissue Type Culture, or other laboratories. The NCI has assembled a three cell-line test and a 60 cell-line test for identifying anti-cancer drugs (see M. R. Boyd and K. D. Paull, Some Practical Considerations and Applications of the NCI in vitro Drug Discovery Screen, Drug Dev. Res. 34:91109, 1995; M. R. Boyd, The NCI In Vitro Anticancer Drug Discovery Screen, Concept, Implementation, and Operation 1985-1995, In Drug Development: Preclinical Screening, Clinical trials and Approval, (Teicher, ed.) Totowa, N.J., Humana Press, 1997, pp. 23-42.

The following examples show that the MT103 family of compounds are effective general anti-cancer agents, and, moreover, that they have selectivity for non-small cell carcinoma cells. The topological computer modeling system described herein was used to generate chemical structures of drugs that are effective against non-small cell carcinoma cells and are active with protein kinase targets, and MT103 was identified as a desirable anti-cancer drug. Subsequent testing by the independent governmental agency NCI provided further evidence that MT103 was an effective anti-cancer drug. Additional experiments with the NCI-H226 cell line provided further proof of the efficacy of MT103. The examples, below, are illustrative and are not intended to be limiting of the invention.

The term heterocyclic is used herein, meaning a cyclic compound having as a ring member at least two different elements. Cyclic compounds may be aromatic or non-aromatic with at least one ring, e.g., one, two, three, or more rings. An aromatic group can be any conjugated ring system containing 4n+2 π-electrons. There are many criteria available for determining aromaticity. A widely employed criterion for the quantitative assessment of aromaticity is the resonance energy. In some embodiments, the resonance energy of the aromatic group is at least 10 KJ/mol. In further embodiments, the resonance energy of the aromatic group is greater than 0 KJ/mol. Aromatic groups may be classified as an aromatic heterocyclic group which contains at least a heteroatom in the 4n+2 π-electron ring, or as an arene or aryl group which does not contain a heteroatom in the 4n+2 π-electron ring. Nonetheless, either the aromatic heterocyclic or the arene or aryl group may have at least one heteroatom in a substituent attached to the 4n+2 π-electron ring. Furthermore, either the aromatic heterocyclic or the arene or aryl group may comprise a monocyclic or polycyclic (such as bicyclic, tricyclic, etc.) aromatic ring. An arene is a monocyclic or polycyclic aromatic hydrocarbon; an aryl is formed by removal of a hydrocarbon from a ring carbon atom of an arene.

Non-limiting examples of the aromatic heterocyclic group are furanyl, thiophenyl, pyrrolyl, indolyl, carbazolyl, benzofuranyl, benzothiophenyl, dibenzofuranyl, dibenzothiophenyl, pyridinyl, pyridazinyl, pyrimidinyl, pyrazinyl, triazinyl, tetrazinyl, petazinyl, quinolinyl, isoquinolinyl, cinnolinyl, phthalazinyl, quinazolinyl, quinoxalinyl, naphthyridinyl, pteridinyl, acridinyl, phenanthridinyl, phenanthrolinyl, anthyridinyl, purinyl, pteridinyl, alloxazinyl, phenazinyl, phenothiazinyl, phenoxazinyl, phenoxathiinyl, dibenzo(1,4)dioxinyl, thianthrenyl, and a combination thereof The aromatic heterocyclic group may also include any combination of the above aromatic heterocyclic groups bonded together either by a bond (as in bicarbazolyl) or by a linking group (as in 1,6 di(10H-10-phenothiazinyl)hexane). The linking group may include an aliphatic group, an aromatic group, a heterocyclic group, or a combination thereof. Furthermore, either an aliphatic group or an aromatic group within a linking group may comprise at least one heteroatom such as O, S, and N. Non-limiting examples of the aryl group are phenyl, naphthyl, benzyl, or tolanyl group, sexiphenylene, phenanthrenyl, anthracenyl, coronenyl, and tolanylphenyl. The aryl group may also include any combination of the above aryl groups bonded together either by a bond (as in biphenyl group) or by a linking group (as in stilbenyl, diphenyl sulfone, an arylamine group). The linking group may include an aliphatic group, an aromatic group, a heterocyclic group, or a combination thereof. Furthermore, either an aliphatic group or an aromatic group within a linking group may comprise at least one heteroatom such as O, S, and N. The term arylamine group includes an (N,N-disubstituted)arylamine group (e.g., diphenylamine, ethylphenylamine, and diethylamine group), a julolidinyl group, and a carbazolyl group.

An alicyclic compound is a cyclic aliphatic compound having at least one ring, e.g., one, two, three, or more rings. The term aliphatic compound refers to an organic compound that is an alkane or alkene or alkyne or their derivative. Examples of alicyclic compounds include cycloalkanes, e.g., cylcobutane, cyclopentane, cylcohexane, cyclooctane, and bicyclo [2.2.1] heptane group. A heterocyclic non-aromatic compound is a compound having at least one ring and at least two different elements in the ring, e.g., an N, O, or S substituted into at least one ring carbon of cylcohexane, cyclooctane, or bicyclo [2.2.1] heptane group.

The term alkyl, unless otherwise specified, refers to a saturated straight, branched, or cyclic hydrocarbon, and specifically includes, e.g., methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The alkyl group can be optionally substituted with any appropriate group, including but not limited to one or more groups selected from halo, hydroxyl, amino, alkylamino, arylarmino, alkoxy, aryloxy, nitro, cyano, sulfonic acid, sulfate, phosphonic acid, phosphate, or phosphonate, either unprotected, or protected as necessary, as known to those skilled in the art. The term alkenyl, unless otherwise specified, is a straight, branched, or cyclic (in the case of C₅₋₆) hydrocarbon with at least one double bond, and may be substituted as described above. The term alkynyl, unless otherwise specified, is a hydrocarbon, straight or branched, with at least one triple bond, and may be substituted as described above. In some embodiments, it is useful to limit the size of these substituents to, e.g., less than about 150, less than about 100, less than about 50, or less than about 20 atoms.

Substitution is liberally allowed on the chemical groups, and on the atoms that occupy a position in a Formula depicted herein, for various physical effects on the properties of the compounds, such as mobility, sensitivity, solubility, compatibility, stability, and the like, as is known generally in the art. In the description of chemical substituents, there are certain practices common to the art that are reflected in the use of language. The term group indicates that the generically recited chemical entity (e.g., alkyl group, alkenyl group, aromatic group, epoxy group, arylamine group, aromatic heterocyclic group, aryl group , alicyclic group, aliphatic group, heterocyclic non-aromatic group etc.) may have any substituent thereon which is consistent with the bond structure of that group. For example, where the term ‘alkyl group’ is used, that term would not only include unsubstituted linear, branched and cyclic alkyls, such as methyl, ethyl, isopropyl, tert-butyl, cyclohexyl, dodecyl and the like, but also substituents having heteroatom such as 3-ethoxylpropyl, 4-(N-ethylamino)butyl, 3-hydroxypentyl, 2-thiolhexyl, 1,2,3-tribromoopropyl, and the like. However, as is consistent with such nomenclature, no substitution would be included within the term that would alter the fundamental bond structure of the underlying group. For example, where a phenyl group is recited, substitution such as 1-aminophenyl, 2,4-dihydroxyphenyl, 1,3,5-trithiophenyl, 1,3,5-trimethoxyphenyl and the like would be acceptable within the terminology, while substitution of 1,1,2,2,3,3-hexamethylphenyl would not be acceptable as that substitution would require the ring bond structure of the phenyl group to be altered to a non-aromatic form because of the substitution. When referring to an epoxy group, the substituent cited includes any substitution that does not destroy the 3-membered ring structure of the epoxy group.

All of these various groups may be optionally derivitized with substituent groups. Suitable substituent groups that may be present on such a “substituted” group include e.g. halogens such as fluoro, chloro, bromo and iodo; cyano; H, hydroxyl group; ester group; ether group; a carbamate, an oxo acid group, an oxocarbon group, an oxo carboxylic acid group, an oxo group, a ketone group; nitro; azido; sulfhydryl; alkanoyl e.g. C₁₋₆ alkanoyl group such as acetyl and the like; carboxamido; alkyl groups, alkenyl and alkynyl groups including groups having one or more unsaturated linkages; alkoxy groups having one or more oxygen linkages; aryloxy such as phenoxy; alkylthio groups; alkylsulfinyl groups; alkylsulfonyl groups; aminoalkyl groups such as groups having one or more N atoms; carbocyclic aryl; aryloxy such as phenoxy; aralkyl having 1 to 3 separate or fused rings; aralkoxy having 1 to 3 separate or fused rings; or a heteroaromatic, heterocyclic, or heteroalicyclic group having 1 to 4 separate or fused rings e.g., with one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl. Other substituents may include groups that include O, S, Se, N, P, Si, C and have between 2 and about 150 atoms. In some embodiments, it is useful to limit the size of any substituent to, e.g., less than about 150, less than about 100, less than about 50, or less than about 20 atoms.

Other suitable substituent groups include these and other N-containing compounds e.g, amines, amides, amidium ions, amine imides, amine oxides, aminium ions, aminonitrenes, nitrenes, aminoxides, nitriles, and nitrile imides. Other suitable substituent groups include these and other S-containing compounds, e.g., sulfonic acid, sulfate, sulfonates, sulfamic acids, sulfanes, sulfatides,sulfenamides, sulfenes, sulfenic acids, sulfenium ions, sulfenyl groups, sulfenylium ions, sulfenyl nitrenes, sulfenyl radicals, sulfides, sulfilimines, sulfimides,sulfimines, sulfinamides, sulfinamidines, sulfines, sulfinic acids, sulfinic anhydrides, sulfinimines, sulfinylamines, sulfolipids, sulfonamides, sulfonamidines, sulfonediimines, sulfones, sulfonic acids, sulfonic anhydrides, sulfonamides, sulfonium compounds, sulfonphthaleins, sulfonylamines, sulfoxides, sulfoximides, sulfoximines, sulfur diimides, thiols, thioacetals, thioaldehydes, thioaldehyde S-oxides, thioanhydrides, thiocarboxylic acids, thiocyanates, thioethers, thiohemiacetals, thioketones, thioketone S-oxides, thiolates, and thionylamines. Other suitable substituent groups include these and other O-containing compounds, e.g., having the form ROH(alcohol), RCOOH (carboxylic acids), RCHO (aldehydes), RR′C═O (ketones), ROR′ (ethers), and RCOOR′ (esters), with the R denoting a bond or atomic element. Other suitable substituent groups include these and other P-containing compounds, e.g., phosphanes, phosphanylidenes, phosphatidic acids, phosphazenes, phosphine oxides, phosphines, phosphinic acids, phosphinidenes, phosphinous acids, phosphoglycerides, phospholipids, phosphonic acids, phosphonitriles, phosphonium compounds, phosphonium ylides, phosphono, phosphonous acids, phosphoramides, and phosphoranes. Carbon is usefull for making substituents and the number of carbons in a heteroatomic structure may be, e.g., between 1 and n−1 when between 2 and n atoms are used to form a substituent with, e.g., O, P, S, or N. In some embodiments, it is usefull to limit the size of these substituents to, e.g., less than about 150, less than about 100, less than about 50, or less than about 20 atoms.

A variety of substituents are contemplated so that some potential combinations of claimed embodiments may be unstable or impractical to make. A person of ordinary skill in the art can select appropriate stable compounds within the disclosed genus of compounds based on the disclosure herein. Therefore, substituents generally are limited to those substituents that result in appropriate valence for the particular substituted element without forming a charged compound or a radical (except for titratable charged groups, stable zwitterionic forms and triplet neutral radicals with formal unpaired spins with full valencies), as can be conventionally determined by a person of ordinary skill in the art.

Introduction to MT103 Family

Formula 1(a) depicts an overall general structure of the MT103 family, with variations being set forth herein In all of the formulas, hydrogen atoms are not necessarily shown and can be presumed as needed at positions appropriate to complete the valence of the associated atoms. MT103 was suggested to be an anticancer agent using a topological computer model and determined to be an anticancer agent using in vitro cell culture experiments. Specifically, MT103 was tested by the NCI, and in other independent tests, and was shown to be an effective anti-cancer drug and an effective inhibitor of cell growth, as described in detail in the Examples below. The same model shows that the MT103 family is generally bioactive and inhibitory of cancer, especially non-small cell lung cancer. Formula 1 shows motifs for the MT103 family, and has been found to be significant with respect to therapeutic function by computer modeling.

An embodiment of the invention is a family of drugs, referred to herein as the MT103 family (Formulas 1-70), that is bioactive, affects cellular functions, and inhibits cancer. A species of this family is depicted as Formula 2, is referred to herein as MT103, and is known as N,N-dicyclohexyl-(1S)-isoborneol-10-sulfonamide, or N, N-dicyclohexyl-2-hydroxy-7,7-dimethylbicyclo[2.2.1]hept-1 -ylmethanesulfonamide. Particular stereoisomers/diastereoisomers are set forth herein; persons of ordinary skill in these arts will appreciate that other stereoisomers/diastereoisomers of these structures, and other structures described herein, are also suitable.

A—Z—Y—X Formula 1(a)

Referring to Formula 1(a), A comprises a bicyclo [2.2.1] heptane group, a heterocyclic group, an alicyclic group, or an aromatic group; Z is a bond or a linking group; Y is a group having one of C, S, O, N, or P; X is —(CH₂)ₙ—X* group, wherein X* is a H, a halogen, a hydroxyl group, a thiol group, a carboxyl group, an amino group, an alkyl group, an alkoxy group, an alkenyl group, an alkynyl group, a heterocyclic group, or an aromatic group; and —(CH₂)ₙ is a group where n is an integer between 1 and about 50, inclusive, and one or more of the methylene groups is optionally replaced by O, S, N, C, B, Si, P, C═O, O═S═O, a heterocyclic group, an aromatic group, an NRₐ group, a CR_{b} group, a CR_{c}R_{d} group or a SiRₑR_{f} where Rₐ, R_{b}, R_{c}, R_{d}, Rₑ, and R_{f} are, each independently, a bond, a pi bond, H, a hydroxyl group, a thiol group, a carboxyl group, a carbamate, an oxocarbon group, an amino group, an amido group, an amide group, a phosphate group, a sulfonate group, an alkyl group, an alkoxy group, an alkenyl group, an alkynyl group, a heterocyclic group, an aromatic group, or a part of a ring group. The groups may be substituted as described herein. A person of ordinary skill in these arts can select appropriately stable compounds from the genera of compounds presented or claimed herein based on conventional chemical principles.

Formula 1(b) depicts a subgenus of Formula 1(a), and Formula 1(c) depicts a bicyclo [2.2.1] heptane group.

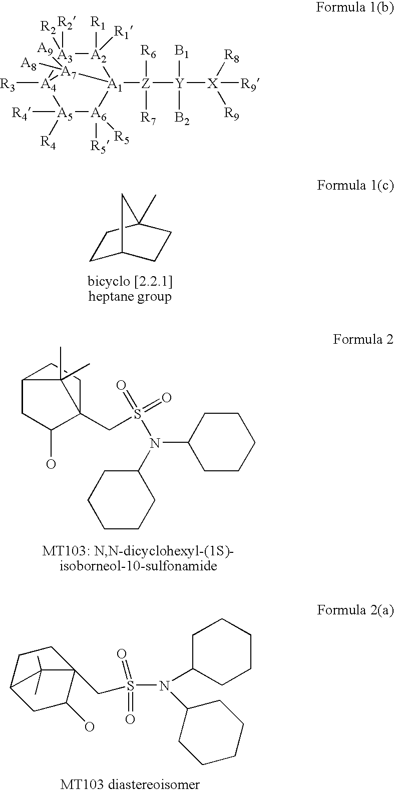

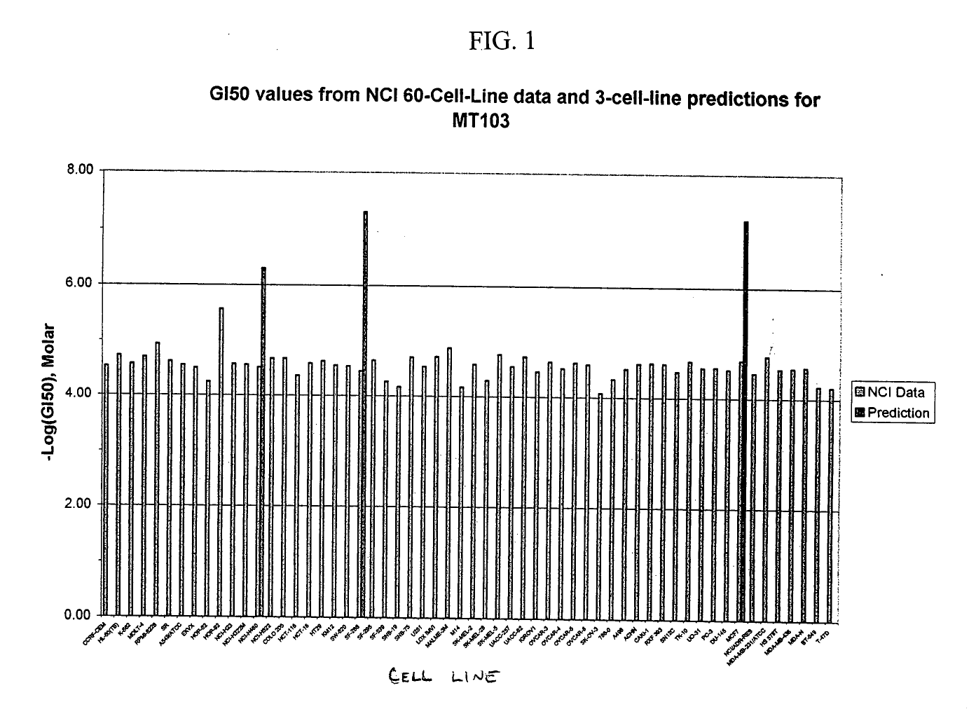

Referring to Formula 1(b), each line joining two occupied positions is a chemical bond, and a line between an occupied position and a vacant position is a nullity, meaning that it is not necessary to posit each position as having an element or group therein, in which case that position and the bind associated therewith, may be considered to be absent form the formula. the positions in FIG. 1(b) potentially being, but not being limited to:

the case wherein A of Formula 1(a) has the formula A₁-A₇ with at least one substituent chosen from R₁-R₅, and R₁′-R₅′; Z has at least one substituent chosen from R₆, and R₇; Y has at least one substituent chosen from B₁ and B2, and X has at least one substituent chosen from R₈, R₉, R₉″, so that the chemical formula is that of Formula 1(b), wherein

A₁-A₇ independently comprise C, S, O, or N; A₈ and A₉ are, each independently, H, an alkyl group, an alkenyl group, an alkynyl group, or a halogen group except that A₈ and A₉ may be combined to form a single group comprising a C or O having a double bond to A₇;

R₉″, B₁, B₂, R₁-R₅, R₁′-R₅′, R₆, and R₇, are independently chosen to be vacant (i.e., a nullity), a lone electron pair, a bond (e.g., a pi bond, a single bond, a double bond, or a triple bond, a bond to an atom or group in another position, e.g., to one of R₉″, B₁, B₂, R₁-R₅, R₁′-R₅′, R₆, and R₇), H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, groups that include O, S, Se, N, P, Si, C and have between 2 and about 150 atoms, or a heterocyclic group; and

R₈ and R₉ comprise at least four atoms.

Further, referring to FIG. 1(b), A₁-A₉, R₈, and/or R₉ may comprise, for example, a cyclic group, a heterocyclic group, an alicyclic group, or a ring having at least one member of the group consisting of S, N, O and P. Examples of substituents are H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. Further, these same groups may be, for example, substituents to at least one of A₁-A₉, R₈, and/or R₉. Persons of skill in these arts will immediately recognize that the list of groups is not limiting and that all combinations or subcombinations of these groups are contemplated.

Further, referring to FIG. 1(b), at least one of R₉″, B₁, B₂, R₁-R₅, R₁′-R₅′, R₆, and R₇ may comprise, for example, a group that is H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. Further, these same groups may be, for example, substituents to at least one of R₉″, B₁, B₂, R₁-R₅, R₁′-R₅′, R₆, and/or R₇. Persons of skill in these arts will immediately recognize that the list of groups is not limiting and that all combinations or subcombinations of these groups are contemplated.

The stereoisomers, e.g., diastereomers, of any of the structures depicted herein are expected to have the functions of the particular structures depicted or described herein. In Formula 2, for example, the chiral group described as “R” could also be “S”. And Formula 2(a) depicts a diastereoisomer of Formula 2.

Various structures are depicted herein as embodiments of the invention. Several subgenera and species within Formula 1(a) are shown. One embodiment is the subgenus shown in Formula 3, which has C₆ alicyclic groups in the positions of R₈ and R₉of Formula 1, a N in position X, an S in position Y, double bonded O in B₁ and B₂, and a saturated C in Z and A₁-A₈. Formulas 4-11 show various embodiments of the subgenus of Formula 3.

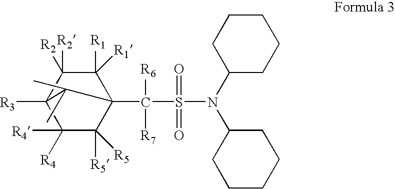

Within the subgenus of Formula 3, species are shown in Formulas 4-11. In all of the formulas below, hydrogen atoms are not shown and can be presumed at all positions appropriate to complete the valence of the associated atoms. In all of these formulas, R₆ and R₇ are either a hydrogen or a methyl group. Also, R₂, R₂′, R₃, R₄ and R₄′ are all hydrogen atoms. The R₁, R₁′, R₅ and R₅′ are as indicated in the specific formula.

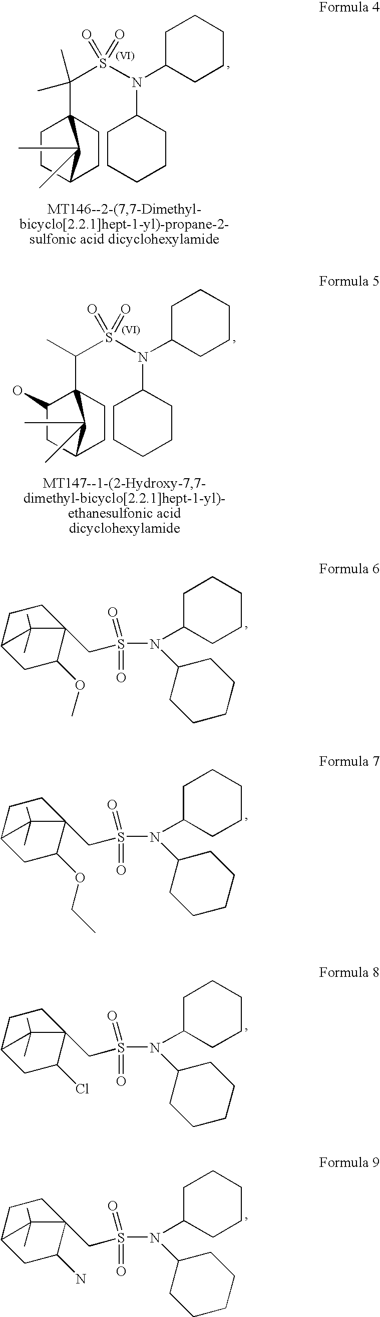

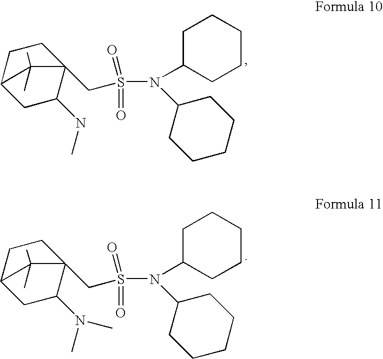

Alternatively, the alicyclic groups of Formula 3 may have various substituents, as described above, and as exemplified in Formulas 12 and 13.

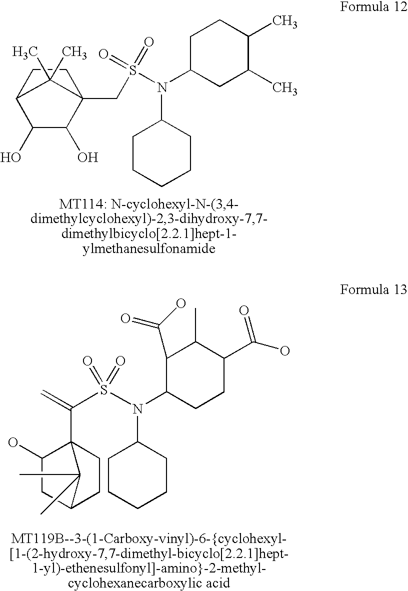

Formulas 14 and 15 show additional embodiments of cyclic groups for substitution into Formula 1, e.g., into the R₈ and R₉ positions. Formula 14 depicts a genus of C₆ alicyclic groups, and Formula 15 depicts a genus of C₆ aryl groups. Potential substituent groups are denoted as having positions T₁-T₆, including T₁′-T₆′. T₁ would be a position in an MT103 structure, e.g., position X in a Formula herein, e.g, Formula 1(a) or 1(b). The chemical groups denoted as T may be, e.g., vacant or a group that is member of the group consisting of a lone electron pair, a bond, a pi bond, H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. Alternatively, e.g., groups that may depend from cyclic structures in an MT103 family molecule include at least one group independently selected from structures consisting of, e.g., H, OH, O, halogens, alkyl (e.g., C₁-C₃), primary amines, secondary amines, tertiary amines, carbonyls, carboxy groups, amides, alkenyl (e.g., C₁-C₃), cycloalkanes, cycloaromatics, alicyclics, unsaturated rings, aromatic rings, alkenyl (e.g., C₁-C₃), alkoxy, and groups of fewer than about 120 atoms having at least one structural element selected from the group consisting O, S, Se, N, P, Si, and C.

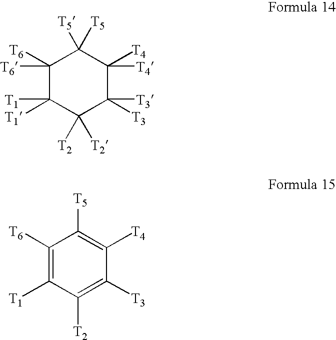

As additional examples of cyclic structures, Formula 16 shows a subgenus of compounds with C₆ aryls, as in Formula 15, substituted into positions R₈ and R₉ of Formula 1(b). Formulas 17-25 show various species within the subgenus of Formula 16.

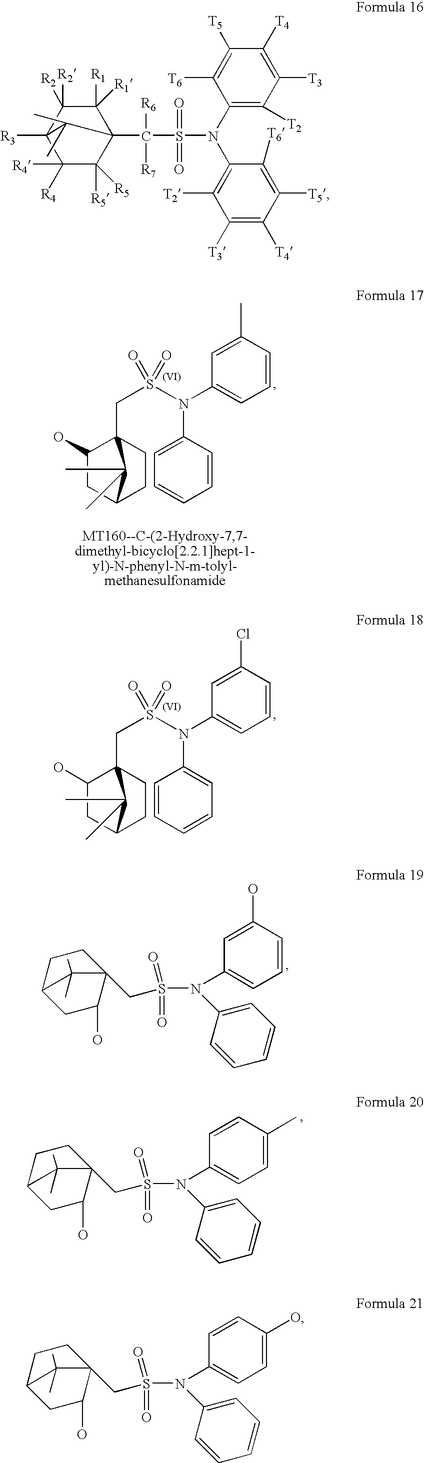

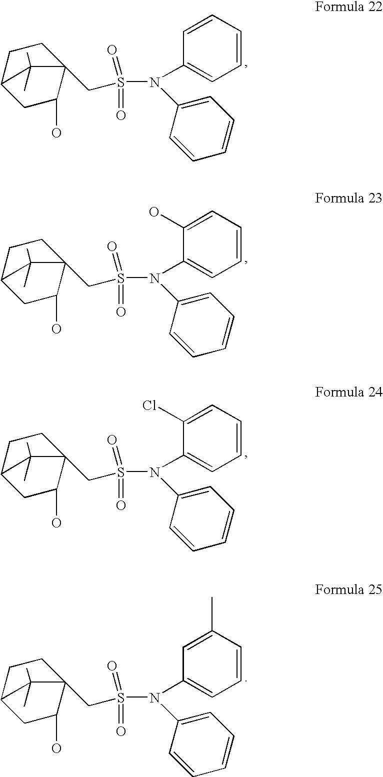

Formula 26 shows an embodiment wherein the T₂ and T₆′ positions of Formula 16 are bonded to the adjacent ring. In general, R₈ and R₉ of Formula 1(b), and other Formulas herein, may have groups that are connected to each other by a bond or a bridge, e.g., having multiple atoms. The various substitutions and substituents described herein may thus include, for example, groups having bonds to groups in other positions as well as unconnected functional groups, as summarized. For example, the positions denoted as T in Formulas 14, 15, and 16 may be filled with groups that join the cyclic structures to each other.

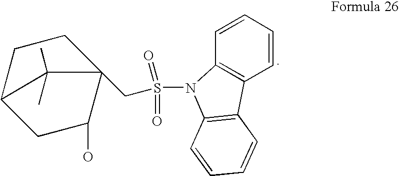

Another subgenus of Formula 1(a) is directed to groups having at least 4 atoms in positions R₈ and R₉of Formula 1(b). Examples of groups for R₈ and/or R₉ in Formula 1(b) are those groups having H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. Formula 27 shows a subgenus having CH₃ groups in positions R₈ and R₉, e.g., with a particular species shown in Formula 28. In other embodiments, the Cn groups may have substituents as described for herein, e.g., for alkyls and alkenyls e.g., as depicted in Formula 29.

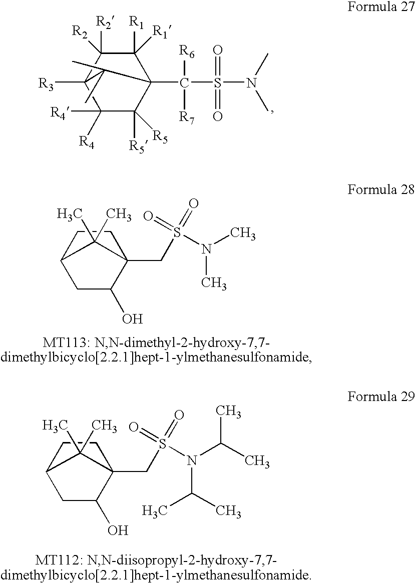

Another subgenus of Formula 1(a) and 1(b) has a cyclic group joined by, e.g., an ether bond to occupy at least one of R₁-R₅ in Formula 1(b); Formula 30 shows an embodiment of FIG. 1(b) comprising an aryl ring joined with an ether linkage at R₁ of Formula 1(b). In Formula 30, R₁′″-R₅′″ may be, e.g., independently chosen to be vacant or a group that is member of the group consisting of a lone pair of electrons, H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. The groups and atoms of Formula 30 may be substituted as described herein. Formulas 31-44 show various species within Formula 30, including substituents substituted as described herein.

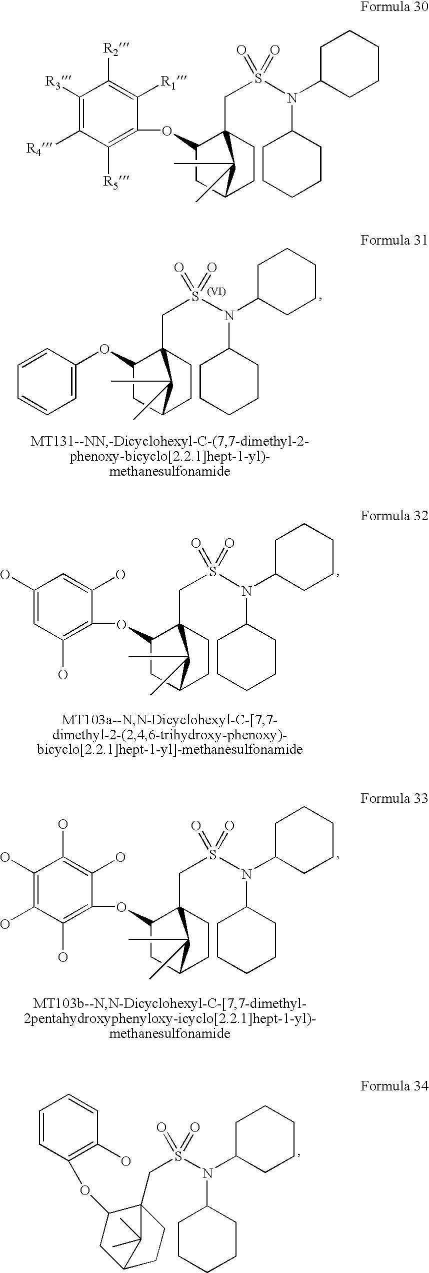

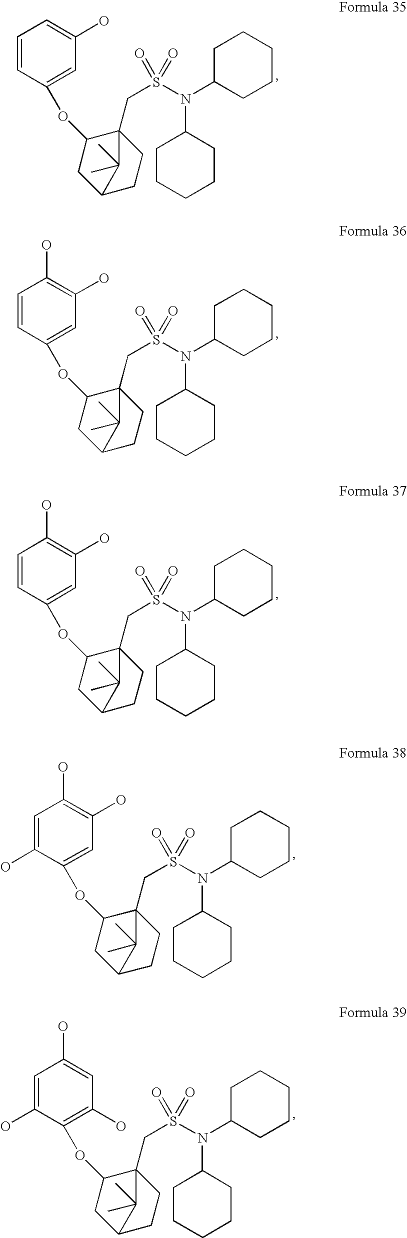

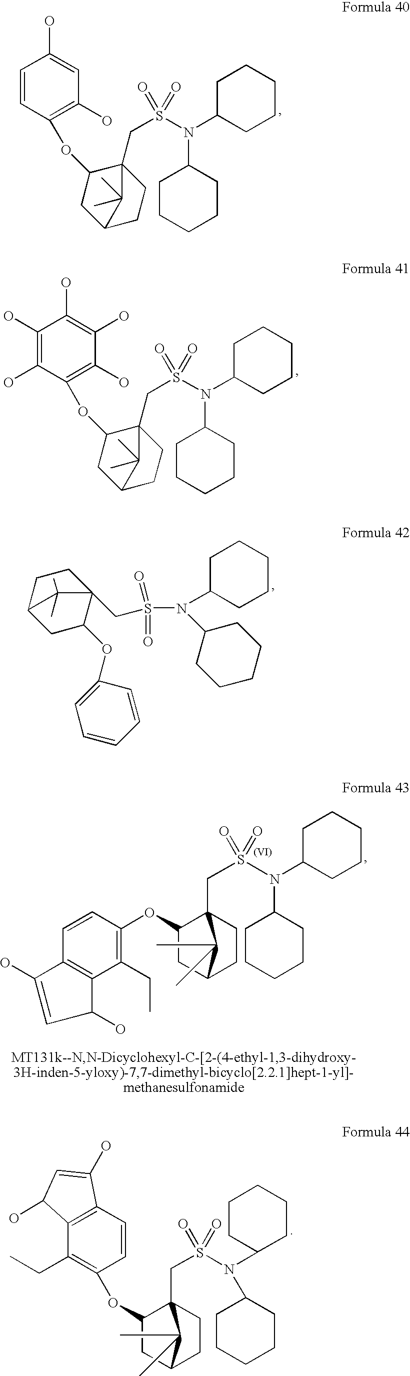

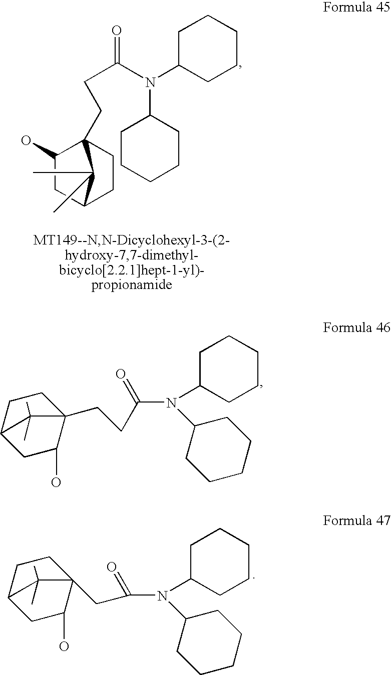

Some structures set forth herein as members of the MT103 family lack a bridge structure. Nonetheless, topological aspects of these molecules indicate inclusion in the MT103 family as being suitable, with the structures providing similar topological or structural motifs. For example, Formula 48-54 depict a group of molecules that do not have a bridge.

Formulas 48 and 49 show examples of subgeneric structures for A in Formula 1(a). These structures may have R₁-R₅, R₁′, R₂′, R₅′, R₅″, and R₅′″ as described for any of R₁-R₅ in Formula 1(b), and may be substituted as described herein. For example, any of R₁-R₅, R₁′, R₂′, R_{5′, R}₅″, and R₅′″, or substituents thereof, may be independently chosen from a lone electron pair, a bond, a pi bond, H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, groups that include O, S, Se, N, P, Si, C and have between 2 and about 150 atoms, or a heterocyclic group. Formulas 50-54 show species of these subgenera as applied to Formula 1(a) or 1(b).

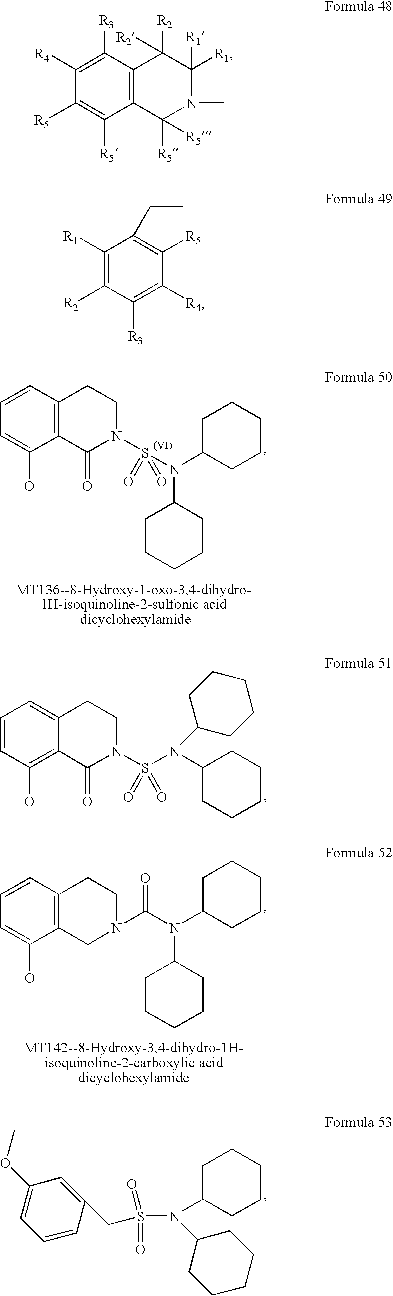

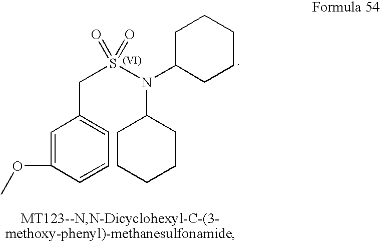

Formula 55, 56 depict subgenera of Formula 1(b) having a N at position X, a S at position Y and an O at position Z; FIG. 56 further depicts a double-bonded O at positions B₁ and B₂. The other positions may be filled as described with reference to Formula 1(a) or 1(b), and include a choice of leaving one or both positions R₆ and R₇ as lone electron pairs. Atoms or groups in the positions of these Formulas may be as those described in Formula 1(b), and groups of these Formulas may be liberally substituted, as described herein.

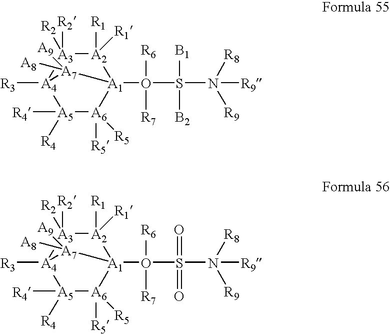

Formula 57-59 depict a subgenus of Formula 1(b) having a N at position X, a S at position Y, a C at position Z, and a C in positions A₁-A₆; Formula 58 further depicts S at position A₇, while Formula 59 depicts N at position A₇. The other positions may be filled as described with reference to Formula 1(b). Atoms or groups in the positions of these Formulas may be as those described in Formula 1(b), and groups of these Formulas may be liberally substituted, as described herein.

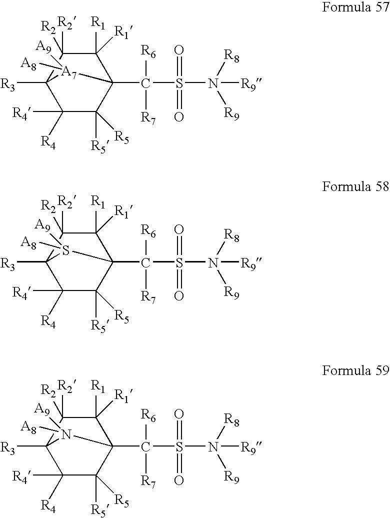

Formulas 60, 61 depict subgenera of Formula 1(b) having a C at position X, and a S at position Y; FIG. 61 further depicts double-bonded O at positions B1 and B₂. The other positions may be filled as described with reference to Formula 1(b). Atoms or groups in the positions of these Formulas may be as those described in Formula 1(b), and groups of these Formulas may be liberally substituted, as described herein. Formulas 62-65 show certain embodiments of an MT103 family member having a C filling position X in Formula 1(b).

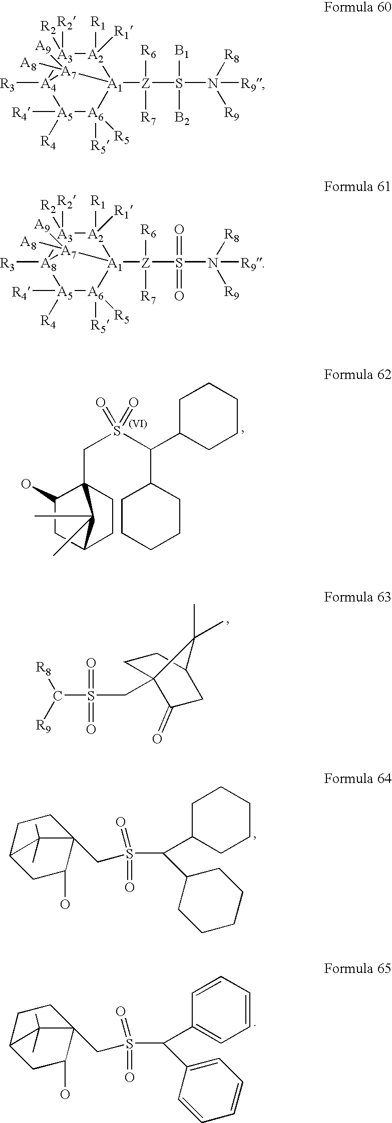

Formula 66 and Formula 67 depict subgenera of Formula 1(a) having a S at position X, and a C at position Y; Formula 67 further depicts C at positions A₁-A₇. The other positions may be filled as described with reference to Formula 1(b), and include a choice of leaving both positions R₆ and R₇ vacant and as lone electron pairs. Atoms or groups in the positions of these Formulas may be as those described in Formula 1(b), and groups of these Formulas may be liberally substituted, as described herein.

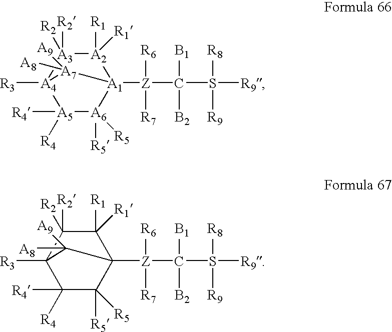

Formulas 68-70 depict a subgenus of Formula 1(b) having cyclic structures in positions R₈ and R₉; Formulas 69, 70 further depict N at position X and S at position Y; Formula 70 further depicts C at positions A₁-A₆. The other positions may be filled as described with reference to Formula 1(b). Positions T, including T₁-T₆, T₁₀-T₁₆, and T₁′-T₁₅′, may be filled as described for any of R₁-R₅ in Formula 1(b). Atoms or groups in the positions of these Formulas may be as those described in Formula 1(b), and groups of these Formulas may be liberally substituted, as described herein.

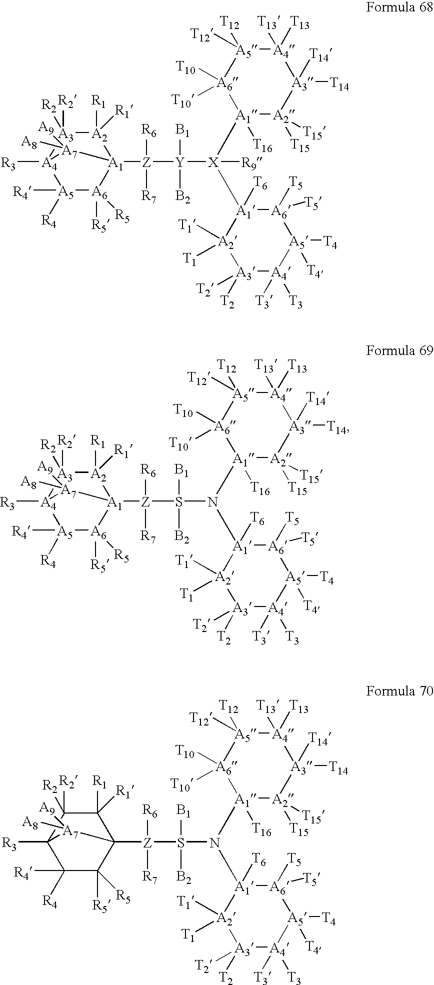

Some chemical groups in MT103 family are believed to be preferable, although other members of the family may have desirable characteristics also. These include hydrogens or short alkyls or alkenyls, particularly methyls, at R₆ and R₇ positions in the Formulas described herein. The C₆ cycloalkyls or their derivatives are considered to be useful at R₈ and R₉. In particular, for R₈ and R₉ such C₆ derivatives that have hydroxyls or carboxyls on at least two positions of the C₆ are useful. The presence of at least one hydroxyl in positions R₁ to R₅ is also believed to be useful, but not essential for function.

Synthesis of Compounds

MT103, as shown in Formula 1, may be purchased from commercially available sources (e.g., ALDRICH, FLUKA, CAS number 99295-72-4) and may be synthesized as described in Chiral auxiliary conferring excellent diastereodifferentiation in reactions of O-enoyl and enolate derivatives, W. Oppolzer, Tetrahedron 43, 1969 (1987) and in W. Oppolzer and Enantioselective systheses of-amino acids from 10-sulfonamido-isobornyl esters and di-t-butyl azodicarboxylate, R. Moretti, Tetrahedron 44, 5541 (1988). Oppolzer taught the use of MT103 as an agent useful for making certain kinds of stereospecific chemicals. Other researchers have published work that describes MT103, and chemically modified derivatives of MT103, as compounds for stereochemical uses.

A variety of reaction schemes are set forth, below. Incorporated herein by reference are textbooks that provide additional details for such reaction scheme: MARCH, J; SMITH, M B. March's Advanced Organic Chemistry: Reactions, Mechanisms, and Structure, 5th Edition; Wiley: New York, 2001; GREENE, T W; WUTS, PGM. Protective Groups in Organic Synthesis, 3rd Edition; Wiley: New York, 1999; LAROCK, R C. Comprehensive Organic Transformations: A Guide to Functional Group Preparations; VCH: New York, 1989; TROST, B M; FLEMING, I (eds.-in-chief). Comprehensive Organic Synthesis: Selectivity, Strategy & Efficiency in Modern Organic Chemistry: Cumulative Indexes, Volume 9; Pergamon Press: Elmsford, N.Y., 1991; D. A. Williams, T. L. Lemke, W. O. Foye, Foye's Principles of Medicinal Chemistry, 15 Feb. 2002, Lippincott Williams & Wilkins Publishers.

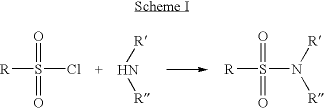

Scheme I depicts a synthesis route for making sulfonamides that can be used to make members of the MT103 family. Referring to Scheme I, aryl or alkyl sulfonyl chloride is reacted with a secondary amine in a single-step reaction. If the R, R′, or R″ contains reactive groups, e.g., amine or thiol, then protective groups may be used to prevent the production of an excessive number of secondary products. Artisans of ordinary skill will be able to synthesize such variants of MT103 as are set forth herein, and other chemicals that are in the family of chemicals that share the features of MT103. References that address sulfonamide synthesis are, for example: Gong, B.; Zheng, C.; Skrzpczak-Jankun, E.; Yan, Y.; Zhang, J. ; J. Am. Chem. Soc. 1998, 120, 11194-11195; Gong, B.; Zheng, C. ;Zeng, H; Zhu, J., J. Am. Chem. Soc. 1999, 121, 9766-9767; Nuckolls C., Hof, F.; Martin, T.; Rebek J Jr.; J. Am. Chem. Soc. 1999, 121, 10281-10285; R. Ohme; H. Preuschhof. Liebigs Ann. Chem. 713, 74-86 (1968); Tetrahedron Letters, 38, 50, 8691-86 (1997); and Bermann, Manfred; Van Wazer, John R. Synthesis (1972), (10), 576-7.

Scheme II presents a general scheme for synthesis of carboxamides. An amine having R′ and R″ may be reacted with an acyl chloride to form a structure as depicted in Scheme II. If R′, or R′, contains, e.g., amine, carboxylic, or hydroxylic groups, then protective groups may be used to prevent the production of an excessive number of secondary products. The protective groups for amine, carboxylic, and hydroxyl groups are described in Carey, Advanced Organic Chemistry, 2^{nd} Ed., Part B, page 539-552 (1983).

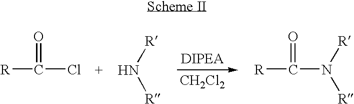

Schemes III(a) and III(b) show the synthesis of members of the MT103 family that contain bicyclic bridges, with MT136, in Formula 50 being used as an example in Scheme III(a). In these scheme, a group, e.g., a bicyclic bridge comprising an amide (Scheme III(a)) or a secondary amine (Scheme III(b)) is derivatized to sulfamoyl chloride by reacting the amide or amine with sulfuryl chloride. The sulfamoyl chloride is reacted with a secondary amine in a single-step reaction. The OCH₃ group is converted to OH in the last step. With respect to reaction of butyl lithium (BuLi) in tetrahydrofuran (THF), the reaction temperature is preferably at about −78° C.

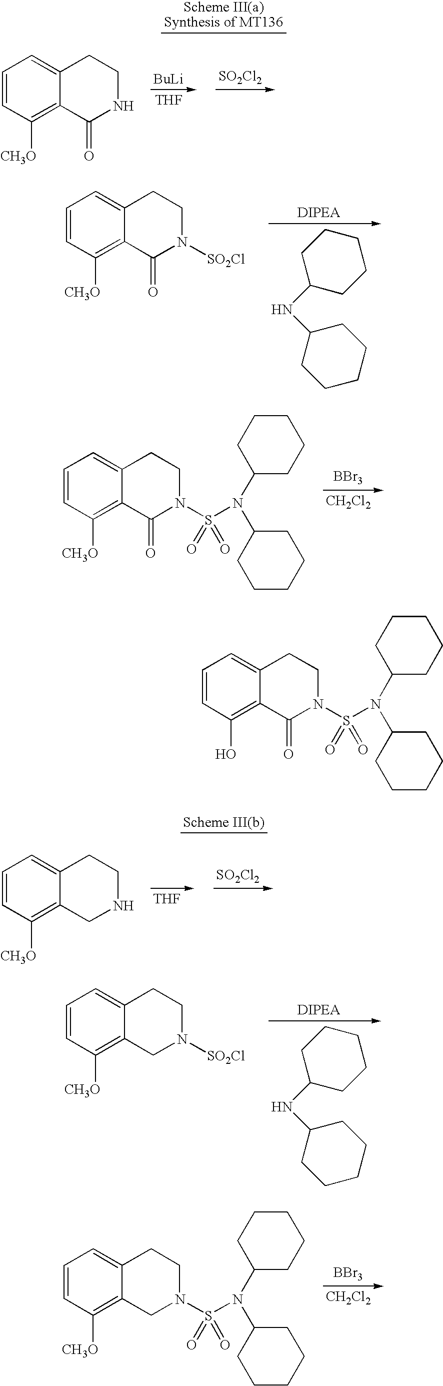

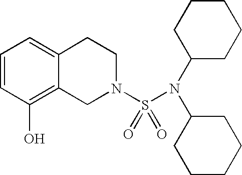

Scheme IV presents a scheme for adding methyl groups to an MT103 family structure, with MT147, in Formula 5, and a variant thereof being used as examples. TBS is t-butyl-dimethyl silyl, LDA is lithium diisopropylamide, Mel is methyl iodide, TBAF is tetrabutyl ammonium fluoride.

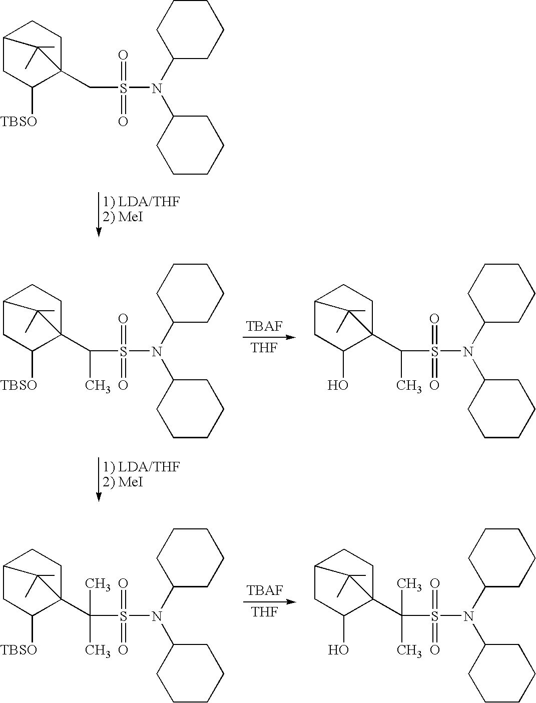

Persons of ordinary skill in these arts can accomplish the synthesis of the precursors needed for making the embodiments of the invention according to Schemes I-IV outlined above, as well as other suitable reactions. Many of the precursors for reaction in the various schemes herein are available commercially and can be readily used or modified, e.g.:

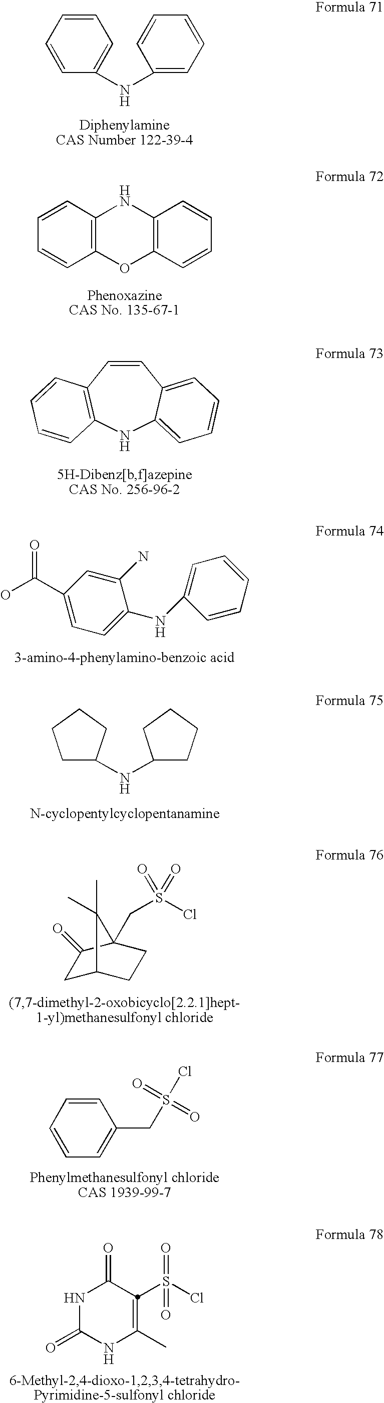

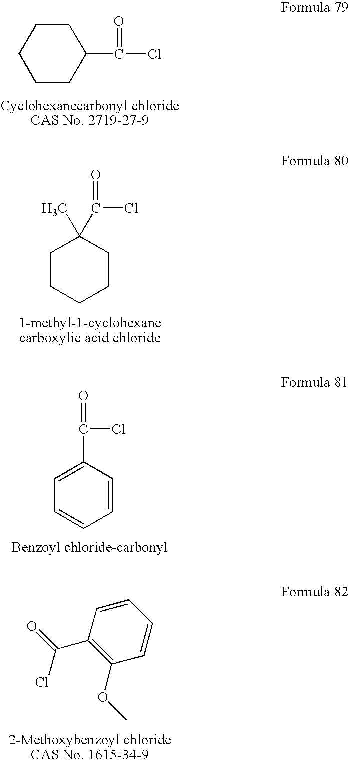

Other embodiments within the MT103 family have the Y group being an oxygen atom. Suitable reactions, for example, involve the formation of an ether linkage —C—O—C—. Persons or ordinary skill in the art recognize that ether linkages can be formed from a sulfuric acid catalyzed de-hydrolysis reaction of two corresponding alcohols or from a reaction between a halide substituted compounds with an alkoxide. In further embodiments, the Y group of the compound is a carbon atom, which can form, for example, a —C—C— linkage. Carbon-carbon bonds can be formed using a Grignard reagent, in which a compound R—C—MgBr reacts with a compound Br—C—R′R″ to form R—C—C—R′R″. Some embodiments are directed to the use of phosphorous in the MT103 family. Various other reaction schemes can be followed by a person of ordinary skill in the art to form the various stable compounds within the MT103 family based on the representative teachings herein.

Phosphorous is multivalent and can form bonds with varying number of atoms (Coordination Number), which can vary from 1 to 6. Phosphorus can form bonds with many other elements and can be substituted into Formulas and reaction schemes as appropriate to satisfy its valency requirements. It has empty d-orbitals which readily accept electrons from donors. In many circumstances, phosphorus can extend its number of bonds to take a new group and via a substitution reaction more readily than carbon. Phosphorus can form bonds readily with oxygen, nitrogen and sulfur, and also can form bonds with carbon. These four bonds enable the linkage of phosphorus to organic compounds to make organophosphorus compounds. References for phosphorous chemistry include, e.g., A Guide to Organophosphorus Chemistry, Louis D. Quin, January 2000 (ISBN: 0-471-31824-8); Organophosphorus Chemistry—A Practical Approach in Chemistry, Edited by Patrick J. Murphy, University of Wales, Bangor, June 2004. Various other reaction schemes can be followed by a person of ordinary skill in the art to form the various stable compounds within the MT103 family based on these and other representative teachings herein.

MT103 Family Compounds

The compounds described herein are designed for activity against non-small cell lung cancer, which is a type of lung neoplasm. To provide some perspective, 95% of primary lung neoplasms are bronchogenic carcinoma/epithelial neoplasms. Bronchogenic carcinoma is commonly divided into two groups: small cell lung cancer, which accounts for about 20% of all cases; and non-small cell lung cancer, which accounts for about 80% of all cases. The non-small cell lung cancer group is further divided into 3 tumor categories based on cell morphology. One category is Squamous cell carcinoma (also called epidermoid carcinoma), which accounts for about 40% of non-small cell lung cancer cases. The second category is Adenocarcinoma, which accounts for 45% of all cases and is the most common lung cancer in non-smokers. The remaining 10% of cases are Large cell lung cancers, which are rapidly fatal.

As shown in Examples 2-5, MT103 has demonstrated in vitro activity against all three categories of non-small cell lung cancer, as demonstrated by tests with multiple cells, including HOP-92 cells (Large cell model), NCI-H460 (Large cell model), NCI-H522 (Adenocarcinoma model), and NCI-H226 cell line (Squamous cell model). As demonstrated by further tests, MT103 is effective against cancer cells in general and is particularly effective against non-small lung cancer cells. In fact, MT103 has a −logGI₅₀ value of 5.6, see FIG. 1 as tested by the NCI in the HOP-92 non-small lung cancer cell line, a large value that indicates that MT103 is particularly effective against this type of cancer, see Table 3. Additional tests conducted by another independent source confirmed this activity, and showed that MT103 has a −logGI₅₀ value of 4.6 for HOP-92, see Table 5 in Example 5. This same series of tests showed that MT103 has a −logGI₅₀ value of 5.1 against the NCI-H226 and 4.1 against NCI-H522, see Table 5 in Example 5. The members of the MT103 family that share motifs of MT103 are therefore also expected to have the anti-cancer function of MT103, as well as its mode of action, and other functions.

Computer modeling and comparison to other chemicals shows that MT103 and the MT103 family are anti-cancer agents, inducers of apoptosis agents, protein kinase agents, hormonal antagonists, antibacterials, antifungals, and hypolipidemics. Examples 1 and 6 show the results of computer models that predict efficacy for the MT103 family. As shown in Examples 1 and 6, chemicals used for such comparisons are paclitaxel, topotecan, etoposide, tamoxifen, anastrozole, and flutamide. Examples 2-5 and 7-8 show that the computer models were successful for predicting the in vitro and in vivo effectiveness of MT103.

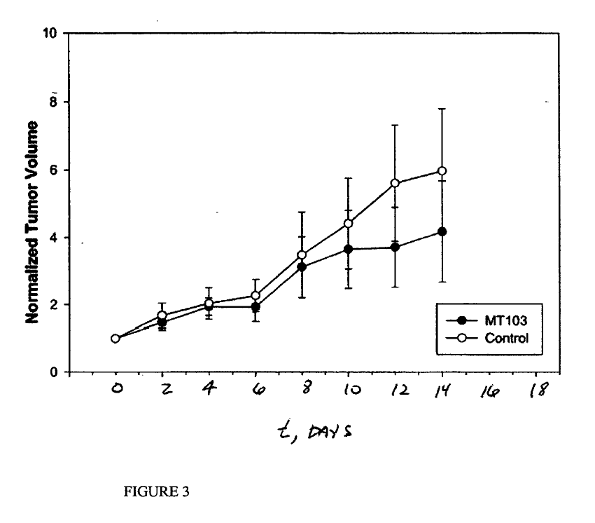

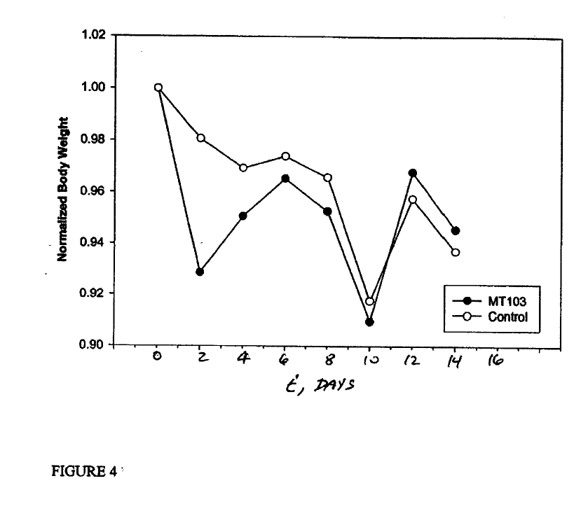

The NOD/SCID induced-tumor mouse model used in Example 7 is a useful predictor of efficacy in human subjects. It showed that MT103 was effective in vivo and was safe. In this model, MT103 slowed down H226 tumor progression, see FIG. 3, and did not cause detectable toxic side effects, see FIG. 4.

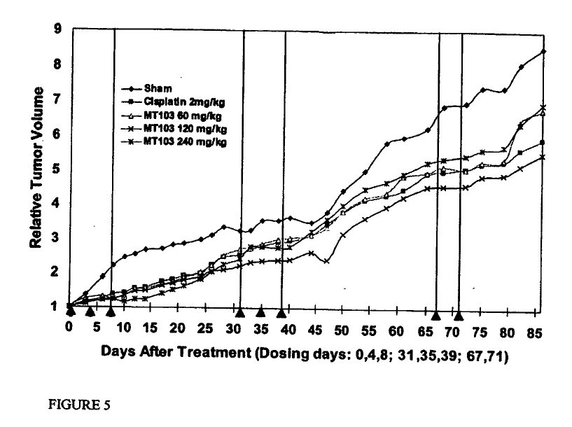

Further tests with this model, using the NCI-H226 squamous carcinoma cell line, as shown in Example 8, showed that MT103 was comparable to, or superior to, the anti-cancer drug cisplatin, see FIG. 5. A range of doses were tested, with 120 mg/kg showing the most improvement compared to cisplatin. Examples 7 and 8 show that MT103 was well tolerated in the animal model and was statistically significantly equivalent, to, or better than, cisplatin at all doses tested.

Since the MT103 family of drugs generally have desirable characteristics, e.g., as outlined in Examples 1 and 6, and as shown by computer modeling and comparisons, they may be used to treat patients to inhibit cancer and to act in the patients as apoptosis agents, protein kinase agents (e.g., PKC-alpha inhibition), hormonal antagonists, antibacterials, antifungals, and hypolipidemic. Cells in vitro and in vivo may be exposed to members of the MT103 family for this purpose. MT103 and the MT103 family can be useful not only as drugs for treating or curing certain cancer types but also as drugs that inhibit certain cancer types in humans and non-human animals. Further, apoptotic agents, hormonal antagonists, antibacterials, and antifungals are important commercial products that are used in many ways; similarly, members of the MT103 family may also be used for such purposes. Accordingly, potential uses would include use for diagnostics, cell testing, and as chemical reagents for commercial sale.

Hypolipidemic drug therapy is used in cases of hyperlidemia (hypercholesterolemia) to reduce cholesterol levels. These drugs have been used in well-controlled studies of patients with high cholesterol levels caused primarily by elevated levels of low-density lipoproteins (LDL). The results of these trials indicate that coronary heart disease (CHD) mortality is reduced by as much as 30% to 40% and that nonfatal events are similarly reduced when hypercholesterolemic patients are treated with moderate doses of hypolipidemic drugs [Scandinavian Simvastatin Survival Study Group, 1994: Shepard et al., 1995; The Long-Term Intervention with Pravastatin in Ischaemic Disease (LIPID) Study Group, 1998].

Further, the MT103 family of chemicals may be used in vitro or in vivo to slow or stop cell growth, kill cells, or to inhibit the growth of cells in vitro or in vivo. Apoptosis inductors, protein kinase agents, and hormonal antagonists are valuable research tools for in vitro and in vivo treatment of cells. Antibacterials and antifungals are valuable products for suppressing, inhibiting and/or killing bacteria and fungi in vitro, in vivo, ex vivo, and in a multitude of environments such as residential, commercial, hospital, and industrial settings. These compounds may be used alone or in combination with other drugs to achieve the most suitable therapy for a patient or other purposes. Appropriate patients include any animals that can benefit from such therapy and include mammals, such as humans, farm animals and pet animals.

Anti-cancer compounds that are effective against one type of cancer can be expected to have an anti-cancer effect against other types of cancers. As shown in Example 3, e.g., Table 3, MT103 displays activity against a wide variety of cancer types. While some compounds described herein may be clinically preferable for use in certain types of cancer, they are also expected to be useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xenoderoma pigmentosum, keratoctanthoma, thyroid follicular cancer and Kaposi's sarcoma. According to another embodiment of the invention, compounds of the invention are directed to therapies for cell proliferative disorders, for example, Alzheimer's disease, viral infections, auto-immune diseases and neurodegenerative disorders.

MT103 and the MT103 family are also useful when delivered in combination with medical devices. For example, the devices may be implantable for a short period of time, or an extended time. Other medical devices are only transiently introduced into the body. Examples of implants made for an extended period of time are stents, e.g., use in blood vessels or other portions of the body, heart valves, pacemakers, defibrillators, angioplasty devices, artificial blood vessels, artificial hearts, and indwelling catheters. Examples of devices implantable for short periods of time include temporary catheters, oxygenator lines, blood pumps, blood filters, and drug delivery systems. Examples of devices introduced only transiently are guidewires, balloons for e.g., angioplasty, and rapidly degradable devices. Other medical devices used with a member of the MT103 family may be devices deployed temporarily, permanently, or semi-permanently in contact with blood, e.g., sensors, biosensors, and diagnostic kits.

One use of MT103 family compounds is to inhibit cell growth around an implanted device. The inhibition may be for a short time, for example while the body's inflammatory reaction is most active, or on a longer term basis. For example, an MT103 family member may be delivered using a strategy of sustained release, slow release, e.g., by enteric coating. The inhibition of cell growth is a significant strategy for the prevention of restenosis after angioplasty or implanting a stent in a blood vessel. Inhibition of cell growth is also a significant strategy for enhancing the biocompatibility of implanted devices so that the reaction of the body to the devices is minimized.

Cells may be exposed to a member of the MT103 family. Exposure can be useful for, e.g., therapeutic treatments, for testing, for diagnosis, and research. The activities of MT103 are useful for studying certain aspects of cellular metabolism and function, e,g., cell growth, or models of disease states such as cancer. Cells is a term used broadly, and includes cells in vitro, in vivo, prokaryotic, eukaryotic, and fungal.

Administration of Compositions

Pharmaceutically acceptable salts of the compounds described herein may be synthesized according to methods known to those skilled in this art, see, for example Pharmaceutical Salts: Properties, Selection, and Use, P. Heinrich Stahl (Editor), Camille G. Wermuth (Editor) June 2002. Generally, such salts are prepared by reacting the free base forms of these compounds with a stoichiometric amount of the appropriate acid in water or in an organic solvent, or in a mixture of the two; generally, nonaqueous media like ether, ethyl acetate, ethanol, isopropanol, or acetonitrile are preferred. Lists of some appropriate salts are found, for example, in Remington's Pharmaceutical Sciences, 17th ed., Mack Publishing Company, Easton, Pa., 1985.

In some embodiments, the compounds described herein are used in combination with one or more potentiators and/or chemotherapeutic agents for the treatment of cancer or tumors. Examples and descriptions of potentiatiors and combination therapies are provided in, for example, U.S. Pat. Nos. 6,290,929 and 6,352,844.

The compounds described herein may be administered as a single active drug or a mixture thereof with other anti-cancer compounds, and other cancer or tumor growth inhibiting compounds. The compounds may be administered in oral dosage forms that include tablets, capsules, pills, powders, granules, elixirs, tinctures, suspensions, syrups, and emulsions. Further, the compounds may be administered in intravenous (bolus or infusion), intraperitoneal, subcutaneous, or intramuscular form.

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used.

Techniques and compositions for making dosage forms useful for materials and methods described herein are described, for example, in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Suitable binders, lubricants, disintegrating agents, coloring agents, flavoring agents, flow-inducing agents, and melting agents may be included as carriers, e.g., for pills. For instance, an active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like.

Suitable binders include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, for example, starch, methyl cellulose, agar, bentonite, xanthan gum, and the like.

The compounds may also be used with liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds may also be coupled to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

The active compounds can be administered orally in solid dosage forms, such as capsules, tablets, and powders, or in liquid dosage forms, such as elixirs, syrups, and suspensions. The active compounds can also be administered parenterally, in sterile liquid dosage forms.

Capsules may contain the active compound and powdered carriers, such as lactose, starch, cellulose derivatives, magnesium stearate, stearic acid, and the like. Similarly, such diluents can be used to make compressed tablets. Both tablets and capsules can be manufactured as immediate release products or as sustained release products to provide for continuous or long-term release of the active compounds. The deliverable form of the compounds can be sugar coated or film coated to mask any unpleasant taste and protect the tablet from the atmosphere, or enteric coated for selective disintegration in the gastrointestinal tract.

For oral administration as a liquid, the drug components may be combined with any oral, non-toxic, pharmaceutically acceptable inert carrier such as ethanol, glycerol, water, and the like. Examples liquid forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Liquid dosage forms may contain, for example, suitable solvents, preservatives, emulsifying agents, suspending agents, diluents, sweeteners, thickeners, and melting agents.

Liquid dosage forms for oral administration can contain coloring and flavoring, as needed. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds described herein may also be administered in intranasal form via use of suitable intranasal vehicles, or via transdermal routes, using those forms of transdermal skin patches known to those skilled in these arts. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

The compounds set forth herein may also be used in pharmaceutical kits for the treatment of cancer, or other purposes, which comprise one or more containers containing a pharmaceutical composition comprising a therapeutically effective amount of the compound. Such kits may further include, if desired, one or more of various components, such as, for example, containers with the compound, containers with one or more pharmaceutically acceptable carriers, additional containers, and instructions. The instructions may be in printed or electronic form provided, for example, as inserts or labels, indicating quantities of the components to be administered, guidelines for administration, and/or guidelines for mixing the components.

Dosage levels include from about 0.1 mg to about 2000 mg of active compound per kilogram of body weight per day are preferable dosages; persons of ordinary skill in these arts will recognize that all doses and ranges between these explicit values are contemplated, e.g., 0.1 to 100, and 1 to 50 mg/kg. The amount of active compound that may be combined with the carrier materials to produce a single dosage form will vary depending upon the host treated and the particular mode of administration. Dosage unit forms will generally contain between from about 0.1 mg to about 10,000 mg of an active compound; persons of ordinary skill in these arts will recognize that all doses and ranges between these explicit values are contemplated. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy. For example, a suitable dosage adopted for oral or intravenous administration of a compound of the MT103 family may range from about 0.1 to about 1000 mg per dose, from 1 to 5 times daily.

The method of administration of the compounds set forth herein can be any suitable method that is effective in the treatment of the particular cancer or tumor type being treated. Treatment may be oral, rectal, topical, parenteral or intravenous administration or by injection into a tumor or cancer. The method of applying an effective amount also varies depending on the disorder or disease being treated. Parenteral treatment may be, e.g., by intravenous, subcutaneous, or intramuscular application of the compounds set forth herein, formulated with an appropriate carrier, additional cancer inhibiting compound or compounds or diluent to facilitate application.

The MT103 compound is commercially available and has been described as useful for certain stereochemical reactions, as referenced in the Synthesis section, herein. Some members of the MT103 family, however, have not been previously disclosed by others. Further, the MT103 family is described herein as having anticancer uses, and as having other biological activities. Embodiments of the invention include compositions that contain a compound as set forth herein, e.g., as in Formulas 1-70.

### EXAMPLES

### Example 1

N,N-dicyclohexyl-(1S)-isoborneol-10-sulfonamide predicted to be an effective anti-cancer agent by topological computer modeling.

Table 1 shows the output for the topological computer model for selected anti-cancer agents and for N,N-dicyclohexyl-(1S)-isoborneol-10-sulfonamide. This output indicates that N,N-dicyclohexyl-(1)-isoborneol-10-sulfonamide is an effective anti-cancer agent. As a control for the computer model, the computer model was also used to predict the results for known anti-cancer agents such as paclitaxel and topotecan, as well as for ifosfamide and Busulfan, agents that are typically not employed as anti-cancer agents. As indicated in Table 1, N,N-dicyclohexyl-(1S)-isoborneol-10-sulfonamide is predicted to be effective for multiple types of cancer, with a −logGI₅₀ value of at least 6.3 for each type of cancer that was tested.

TABLE 1
Topological computer model results for MT103
and selected anti-cancer compounds.

| Modeled | | | | | |
|---|---|---|---|---|---|
| Properties for | | | | | |
| Compounds | MT103 | Paclitaxel | Topotecan | Ifosfamide | Busulfan |
| Activity | >90%* | >90%* | >90%* | <10% | <10% |
| against breast | | | | probability | probability |
| cancer | | | | | |
| NCI-MCF7 | | | | | |
| -log(GI₅₀), | 7.2 | 8.8 | 7.5 | <5 | <5 |
| molar | | | | | |
| Activity | >90%* | >90%* | >90%* | <10% | <10% |
| against lung | | | | probability | probability |
| cancer | | | | | |
| NCI-H460 | | | | | |
| -log(GI₅₀), | 6.3 | 7.4 | 7.6 | <5 | <5 |
| molar | | | | | |
| Activity | >90%* | >90%* | >90%* | <10% | <10% |
| against CNS | | | | probability | probability |
| cancer | | | | | |
| NCI-SF268 | | | | | |
| -log(GI₅₀), | 7.3 | 7.6 | 7.0 | <5 | <5 |
| molar | | | | | |
| Protein Kinase | 0.9 | 0.1 | 2 | >4 | >4 |
| C Inhibitor, | | | | | |
| Log Ki, nM | | | | | |
| Induction of | 21% | 69% | 3.6% | 0% | 0% |
| Apoptosis | | | | | |

The pharmacokinetic properties of MT103 have been calculated and result in some predictions that show the usefullness of the chemical. The predictions indicate that MT103 will decay according to a 2 or 3 compartment model with a predicted terminal elimination half-life of about 3 hours. An average peak plasma concentration of about 1 mg/L should occur about an hour after dosing. The total clearance is estimated to be about 25 L/h and the mean apparent volume of distribution at steady state as about 1.5 L/kg. The expected mean oral bioavailability of MT103 is about 20% and about 79% of the MT103 in the plasma is bound to protein in the body. Analogs having a structure similar to MT103 are expected to have similar pharmacokinetic properties.

### Example 2

NCI three cell-line test indicates that N,N-dicyclohexyl-(1S)-isoborneol-10-sulfonamide is an effective anti-cancer agent.

This Example shows that MT103 is predicted by in vitro cell testing to be an effective anti-cancer agent. The testing in this Example was performed by NCI, as per their 3-cell line panel test. The results are reported as the percent of the growth of the treated cells compared to the untreated control cells. The criterion for being an effective compound and for being subjected to further testing is that the tested compound reduce the growth of any one of the three cell lines to approximately 32% or less. As shown in Table 2, MT103 was much more effective than the commonly accepted scientific accepted criterion; in fact, MT103 reduced the growth of all three cell lines to 16% or less at the one concentration tested.

TABLE 2
MT103 shown to be effective by NCI 3 cell-line test.

| | Growth, Percentage | | |
|---|---|---|---|
| Concentration | MCF7 | NCI-H460 | SF-268 |
| of MT103 in | Cell line | Cell Line (Non- | Cell Line (Central |
| growth medium | (Breast Cancer) | Small cell Lung) | Nervous System) |
| 1(10⁻⁴) Molar | 0 | 16 | 14 |

The methods for conducting the test are described below in Example 3, except that the cells were exposed to a single concentration of MT103, at 1×10⁻⁴ Molar, and colorimetric determinations were made with alamar blue (Biotechniques 21(5):780-782 (1996)).

### Example 3

NCI sixty cell line test shows that MT103 is an effective anti-cancer drug.

The NCI tested MT103 with 60 cell lines, and reported the G150, TGI, and LC₅₀ values of MT103 for each cell line, see FIG. 1 and Table 3.

TABLE 3
NCI 60 cell-line test for the drug MT103.

| | | | |
|---|---|---|---|
| Panel/Cell Line | G150 | TGI | LC50 |

| | | | |
|---|---|---|---|
| Leukemia | | | |
| CCRF-CEM | 2.97E−05 | >1.00E−04 | >1.00E−04 |
| HL-60 (TB) | 1.90E−05 | 8.33E−05 | >1.00E−04 |
| K-562 | 2.68E−05 | >1.00E−04 | >1.00E−04 |
| MOLT-4 | 2.06E−05 | >1.00E−04 | >1.00E−04 |
| RPMI-8226 | 1.18E−05 | 3.68E−05 | >1.00E−04 |
| SR | 2.38E−05 | >1.00E−04 | >1.00E−04 |
| Non-Small Cell Lung Cancer | | | |
| A549/ATCC | 2.83E−05 | >1.00E−04 | >1.00E−04 |
| EKVX | 3.13E−05 | 9.83E−05 | >1.00E−04 |
| HOP-62 | 5.75E−05 | >1.00E−04 | >1.00E−04 |
| HOP-92 | 2.75E−06 | 3.16E−05 | >1.00E−04 |
| NCI-H23 | 2.69E−05 | 8.31−E05 | >1.00E−04 |
| NCI-H322M | 2.73E−05 | >1.00E−04 | >1.00E−04 |
| NCI-H460 | 3.09E−05 | >1.00E−04 | >1.00E−04 |
| NCI-H522 | 2.15E−05 | 5.50E−05 | >1.00E−04 |
| Colon Cancer | | | |
| COLO 205 | 2.13E−05 | 4.98E−05 | >1.00E−04 |
| HCT-116 | 4.38E−05 | >1.00E−04 | >1.00E−04 |
| HCT-15 | 2.57E−05 | >1.00E−04 | >1.00E−04 |
| HT29 | 2.35E−05 | >1.00E−04 | >1.00E−04 |
| KM12 | 2.84E−05 | 9.73E−05 | >1.00E−04 |
| SW-620 | 2.88E−05 | >1.00E−04 | >1.00E−04 |
| CNS Cancer | | | |
| SF-268 | 3.58E−05 | >1.00E−04 | >1.00E−04 |
| SF-295 | 2.26E−05 | >1.00E−04 | >1.00E−04 |
| SF-539 | 5.46E−05 | >1.00E−04 | >1.00E−04 |
| SNB-19 | 6.80E−05 | >1.00E−04 | >1.00E−04 |
| SNB-75 | 1.95E−05 | 8.83E−05 | >1.00E−04 |
| U251 | 2.87E−05 | >1.00E−04 | >1.00E−04 |
| Melanoma | | | |
| LOX IMVI | 1.90E−05 | 3.95E−05 | 8.23E−05 |
| MALME-3M | 1.31E−05 | 4.44E−05 | >1.00E−04 |
| M14 | 6.78E−05 | >1.00E−04 | >1.00E−04 |
| SK-MEL-2 | 2.60E−05 | 6.77E−05 | >1.00E−04 |
| SK-MEL-28 | 5.07E−05 | >1.00E−04 | >1.00E−04 |
| SK-MEL-5 | 1.68E−05 | 3.61E−05 | 7.77E−05 |
| UACC-257 | 2.85E−05 | 9.53E−05 | >1.00E−04 |
| UACC-62 | 1.88E−05 | 4.65E−05 | >1.00E−04 |
| Ovarian Cancer | | | |
| IGROV1 | 3.48E−05 | >1.00E−04 | >1.00E−04 |
| OVCAR-3 | 2.30E−05 | 6.30E−05 | >1.00E−04 |
| OVCAR-4 | 3.02E−05 | >1.00E−04 | >1.00E−04 |
| OVCAR-5 | 2.37E−05 | 5.70E−05 | >1.00E−04 |
| OVCAR-8 | 2.53E−05 | >1.00E−04 | >1.00E−04 |
| SK-OV-3 | 8.32E−05 | >1.00E−04 | >1.00E−04 |
| Renal Cancer | | | |
| 786-0 | 4.63E−05 | >1.00E−04 | >1.00E−04 |
| A498 | 3.04E−05 | 9.07E−05 | >1.00E−04 |
| ACHN | 2.38E−05 | >1.00E−04 | >1.00E−04 |
| CAKI-1 | 2.35E−05 | 9.05E−05 | >1.00E−04 |
| RXF 393 | 2.42E−05 | 9.77E−05 | >1.00E−04 |
| SN12C | 3.30E−05 | >1.00E−04 | >1.00E−04 |
| TK-10 | 2.16E−05 | 9.02E−05 | >1.00E−04 |
| UO-31 | 2.80E−05 | 8.94E−05 | >1.00E−04 |
| Prostate Cancer | | | |
| PC-3 | 2.78E−05 | >1.00E−04 | >1.00E−04 |
| DU-145 | 3.05E−05 | >1.00E−04 | >1.00E−04 |
| Breast Cancer | | | |
| MCF7 | 2.06E−05 | 9.05E−05 | >1.00E−04 |
| NCI/ADR-RES | 3.49E−05 | >1.00E−04 | >1.00E−04 |
| MDA-MB-231/ATCC | 1.69E−05 | 4.71E−05 | >1.00E−04 |
| HS 578T | 2.90E−05 | >1.00E−04 | >1.00E−04 |
| MDA-MB-435 | 2.84E−05 | 9.88E−05 | >1.00E−04 |
| MDA-N | 2.71E−05 | >1.00E−04 | >1.00E−04 |
| BT-549 | 5.93E−05 | >1.00E−04 | >1.00E−04 |
| T-47D | 6.20E−05 | >1.00E−04 | >1.00E−04 |

Methodology: The NCI conducted a test of the MT103 drug against 60 human cell lines, with a minimum of five concentrations of MT103 at 10-fold dilutions. A 48 hour continuous drug exposure was used, and a sulforhodamine B (SRB) protein assay was used to estimate cell viability and growth. The human tumor cell lines of the cancer screening panel were grown in RPMI 1640 medium containing 5% fetal bovine serum and 2 mM L-glutamine. Cells were inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air, and 100% relative humidity for 24 h prior to addition of experimental drugs.

After 24 h, two plates of each cell line were fixed in situ with TCA, to represent a measurement of the cell population for each cell line at the time of drug addition (Tz). MT103 was solubilized in dimethyl sulfoxide at 400-fold the desired final maximum test concentration and stored frozen prior to use. At the time of drug addition, an aliquot of frozen concentrate was thawed and diluted to twice the desired final maximum test concentration with complete medium containing 50 μg/ml gentamicin. Additional four, 10-fold or ½ log serial dilutions were made to provide a total of five drug concentrations plus control. Aliquots of 100 μl of these different drug dilutions were added to the appropriate microtiter wells already containing 100 μl of medium, resulting in the required final drug concentrations.

Following drug addition, the plates were incubated for an additional 48 h at 37° C., 5% CO₂, 95% air, and 100% relative humidity. For adherent cells, the assay was terminated by the addition of cold TCA. Cells were fixed in situ by the gentle addition of 50 μl of cold 50% (w/v) TCA (final concentration, 10% TCA) and incubated for 60 minutes at 4° C. The supernatant was discarded, and the plates were washed five times with tap water and air dried. Sulforhodamine B (SRB) solution (100 μl) at 0.4% (w/v) in 1% acetic acid was added to each well, and plates were incubated for 10 minutes at room temperature. After staining, unbound dye was removed by washing five times with 1% acetic acid and the plates were air-dried. Bound stain was subsequently solubilized with 10 mM trizma base, and the absorbance was read on an automated plate reader at a wavelength of 515 nm. For suspension cells, the methodology was the same except that the assay was terminated by fixing settled cells at the bottom of the wells by gently adding 50 μl of 80% TCA (final concentration, 16% TCA). Using the seven absorbance measurements (time zero, (Tz), control growth, (C), and test growth in the presence of drug at the five concentration levels (Ti)), the percentage growth was calculated at each of the drug concentrations levels. Percentage growth inhibition was calculated as:

[(Ti-Tz)/(C-Tz)]×100 for concentrations for which Ti>/=Tz

[(Ti-Tz)/Tz]×100 for concentrations for which Ti<Tz.

Three dose response parameters were calculated for each experimental agent. Growth inhibition of 50% (GI50) was calculated from [(Ti-Tz)/(C-Tz)]×100=50, which is the drug concentration resulting in a 50% reduction in the net protein increase (as measured by SRB staining) in control cells during the drug incubation. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment was calculated from [(Ti-Tz)/Tz]×100=−50. Values were calculated for each of these three parameters if the level of activity is reached; however, if the effect was not reached or was exceeded, the value for that parameter was expressed as greater or less than the maximum or minimum concentration tested.

### Example 4

N,N-dicyclohexyl-(1S)-isoborneol-10-sulfonamide is an effective inhibitor of the cell growth of the cancerous cell line NCI-H226.

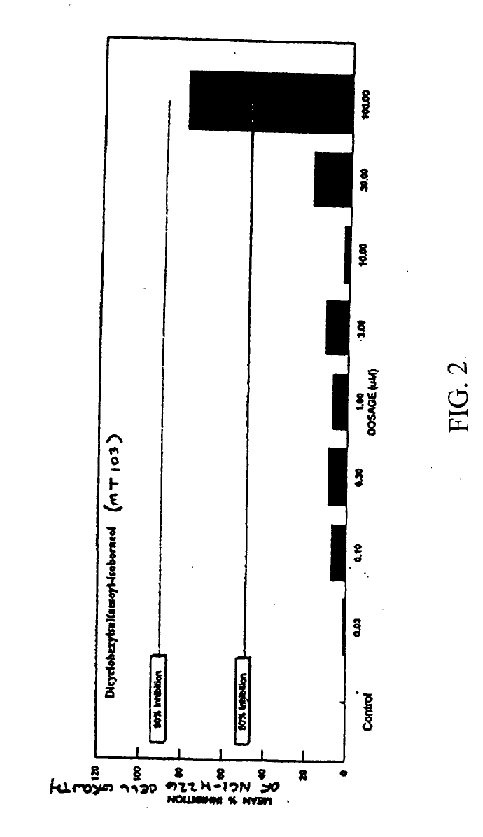

This Example shows that MT103 is a drug for treating human cancer, particularly non-small cell lung cancer. MT103 was tested with the NCI-H226 human non-small cell lung cancer cell line, and effectively inhibited growth of the cancer, see FIG. 2. The GI₅₀ for MT103 was 66 μM.

Methods The MTS assay was employed in the evaluation of these compounds. The cells were harvested, centrifuged to remove the medium, and suspended in fresh complete medium. Samples were taken to determine cell density. The cell count was determined with a Coulter Model Z cell counter and viability was measured with propidium iodine staining call by analysis on Coulter EPICS flow cytometer. The cell line was plated at 5×10³ cells per well in complete medium. On the following day, the cells were closed with the dilutions of the compound. The plates were analyzed on Day for after initiation of treatment.

The cell line was propagated using standard tissue culture procedures and seeded in microtiter plates prior to dosing. Control groups included a mock treatment, media control, and a positive control (doxorubicin, 1 μM). For each concentration level, eight replicates were treated. The cell line was propagated under sterile conditions at 37° C. in 5% CO₂ and 95% humidity. MT103 was stored at 4° C. until dissolved and diluted in complete medium.

Anti-cellular effects of the compound were assessed with the MTS dye conversion assay. MTS was purchased as a single solution, and stored at −20° C. Sample wells were treated with 20 microliters of the MTS solution and the plates were incubated for four hours at 37° C. to allow for conversion into the liquid soluble formazan product. The absorbance of formazan in each monolayer was measured at 490 nm on a Coulter microplate reader at four hours after addition of the MTS.

### Example 5

Dosage levels for human use.

These results show the activity of MT103 at dose levels suitable for human use and assess anticellular activity. Testing was conducted employing standard tissue culture techniques and an anchorage-independent colony-forming assay.

MT103 was tested against the lung carcinoma cell lines NCI-H226, NCI-H522, and HOP-92. An anchorage-independent colony-forming assay was employed. For the experiment, the cells were harvested, centrifuged to remove the media, and suspended in Iscove's Modified Dulbecco's Medium (IMDM). Samples were taken to determine cell density. The cell count was determined with a Coulter Model Z₁ cell counter and viability was measured with propidium iodide staining followed by analysis on a Coulter EPICS XL flow cytometer. Each cell line was plated at 1×10⁵ cells per dish (35 mm) in IMDM with 20% FBS and 1% methylcellulose containing the appropriate concentration of test compound or DMSO control. Five concentrations of each compound and a DMSO control were tested in each of the three cell lines. The colonies per plate were counted on Day 14 after the initiation of treatment. The cell lines were propagated under sterile conditions and incubated at 37° C. in HEPA-filtered CO₂ tissue culture incubators with 5% CO₂ and 95% humidity. The cell line was subcultured weekly to bi-weekly and used in experiments. The test compounds were kept refrigerated under light protected conditions until dissolved in DMSO.

Anticellular effects of the compounds on the tumor line were assessed with a colony-forming assay. Briefly, cell were harvested and adjusted to 3×10⁶ viable cells/mL in IMDM. Compounds (200×) were prepared in DMSO and serially diluted in DMSO to give appropriate testing concentrations. Cells (100 μL), compound or DMSO control (16.5 μL) and IMDM (183.5 μL) were added to the appropriate assay tubes (12×75 mm) containing 3 mL of complete media, IMDM with 20% FBS and 1% methylcellulose. Tubes were then vortexed and 1.1 mL of the cell suspension was added to each of two 35 mm dishes. These dishes were then incubated for 14 days in HEPA-filtered CO₂ tissue culture incubators with 5% CO₂ and 95% humidity. The colonies in each dish were then counted and the duplicates were averaged and reported

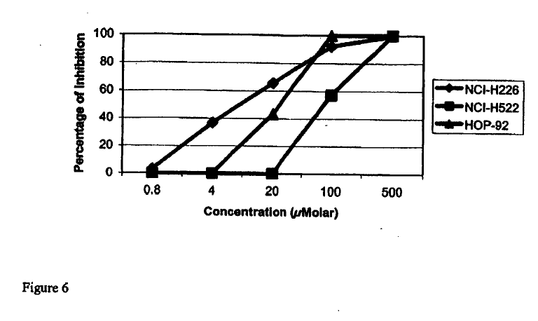

Results for this study are presented in the tables below, and in FIG. 6.

TABLE 4
Summary of Percent Inhibition

| | Concentration | % Inhibition | | |
|---|---|---|---|---|
| Compound | (μM) | NCI-H226 | NCI-H522 | HOP-92 |
| MT103 | 0.8 | 3.1 | 0 | 0 |
| | 4 | 36.7 | 0 | 0 |
| | 20 | 65.6 | 0 | 43.3 |
| | 100 | 92.2 | 57.2 | 100 |
| | 500 | 99.7 | 100 | 100 |

TABLE 5
Summary of Inhibitory Concentration (IC₅₀)

| | IC₅₀ (μM) | | |
|---|---|---|---|
| Compound | NCI-H226 | NCI-H522 | HOP-92 |
| MT103 | 8.39 | 81.66 | 24.19 |

### Example 6

Analogs of MT103 determined to be effective therapeutic agents.

Results of the topological computer model showed that members of the MT103 family are effective therapeutic agents. Table 6 shows compounds tested with the computer model and determined to be efficacious. MT103 is N,N-dicyclohexyl-(1S)-isoborneol-10-sulfonamide. Analog B is N-cylohexyl-N-(3,4-dimethylcyclohexyl)-2,3-dihydroxy-7,7-dimethylbicyclo[2.2.1]hept-1-ylmethanesulfonamide. Analog C is N1-cyclohexyl-N1-{4-[(E)ethylidene]-3- methylenecycolhexy}-1-(2-hydroxy-7,7-dimethylbicyclo[2.2.1]hept-1-yl)-1-ethylenesulfonamide. Analog D is 4-cyclohexyl[1-(2-hydroxy-7,7-dimethylbicyclo[2.2.1]hept-1-yl)vinyl]sulfanamido-2-methyl-1,3 cyclohexanedicarboxylic acid. Analog E is 4-[3,4-dihydroxycyclohexy1(2-hydroxy-7,7-dimethylbicyclo[2.2.1]hept-1-ylmethyl)sulfonamido]-2-methyl-1,3-cyclohexanedicarboxylic acid.

TABLE 6

| | Species of MT103 family tested by computer modeling | | | | | |
|---|---|---|---|---|---|---|
| | and determined to be efficacious | | | | | |
| Compound | MT103 | Analog A | Analog B | Analog C | Analog D | Analog E |
| Acetyl cholinesterase | >90%* | >90%* | >90%* | >90%* | >90%* | >90%* |
| inhibitor | | | | | | |
| Peak time (hours) | 1 | 2 | 2 | 2 | 2 | 2 |
| Peak concentration | 1 | 0.04 | 2 | 1 | .1 | 0.4 |
| (mg/L) | | | | | | |
| Metabolites | 26 | 8 | 59 | 95 | 74 | 40 |
| (% of hepatic | | | | | | |
| elimination) | | | | | | |
| Activity against | >90%* | >90%* | >90%* | >90%* | >90%* | >90%* |
| breast cancer | (7.2) | (7.0) | (7.3) | (7.3) | (7.4) | (7.6) |
| NCI-MCF7 | | | | | | |
| (-log(GI50), molar) | | | | | | |
| Activity against | >90%* | >90%* | >90%* | >90%* | >90%* | >90%* |
| lung cancer | (6.3) | (6.3) | (6.4) | (6.6) | (6.6) | (6.8) |
| NCI-H460 | | | | | | |
| (-log(GI50), molar) | | | | | | |
| Activity against | >90%* | >90%* | >90%* | >90%* | >90%* | >90%* |
| CNS cancer | (7.3) | (7.0) | (7.4) | (7.4) | (7.5) | (7.5) |
| NCI-SF268 | | | | | | |
| (-log(GI50), molar) | | | | | | |
| Induction of | 21 | 25 | 40 | 40 | 31 | 35 |
| apoptosis | | | | | | |
| (%) | | | | | | |
| Log Ki (nM) | 0.9 | 1.5 | 0.8 | 1.0 | 1.0 | 0.5 |
| for inhibitors of | | | | | | |
| Protein Kinase-C | | | | | | |

*These percentages indicate calculated probabilities that the compound will have the indicated function.

### Example 7

In vivo test data showing safety

This experiment provides in vivo test data showing that the MT103 family of compounds are safe and well tolerated, as measured by weight control of the test subjects, and were effective anti-cancer agents over the duration of the test. MT103 slowed down H226 tumor progression significantly in NOD/SCID mice, (NOD.CB₁₇-Prkdcscid/J) without any evidence of toxicity. The change that occurred with the treatment is greater than would be expected by chance (p=0.038).

A standard induced-tumor NOD/SCID mouse model was used to perform this investigation, with the mouse model being based on protocols for human xenograft systems developed at the National Cancer Institute. H226 cell cancer tumors were induced in NOD/SCID mice; tumors at sizes approximately 100 mm³ were treated with 30 mg/kg MT103 or a control containing only vehicle (2.5% DMSO in distilled water) administered in intraperitoneal injections every other day for two weeks. As shown in FIG. 4, MT103 did not have deleterious effect on mice body weight. FIG. 3 shows that tumor volume was significantly decreased.

### Example 8

In vivo test data showing efficacy.

This experiment provides in vivo test data showing that the MT103 family of compounds are effective anti-cancer agents. Comparison to the anti-cancer drug cisplatin showed that MT103 had a comparable or superior effect, depending upon the dosages administered. MT103 was tested at three dose levels (60, 120, and 240 mg/kg). Experiments were performed as described in Example 7, NOD/SCID mice, (NOD.CB₁₇-Prkdcscid/J) except that tumors were treated with: one of three doses of MT103 or Cisplatin or sham for 3 intraperitoneal injections (one every 4 days) in a 2-cycle 56-day test that was extended to another cycle to 86 days, as reflected in FIG. 5. Tumor volumes were measured every other day. As shown in FIG. 5, mean tumor growth slowed in mice receiving MT103, which demonstrated significant efficacy (p=0.046). There were no observable differences in organ appearance in control versus animals treated with 120 mg/kg MT103. Also, there were no differences in blood counts and electrolytes between these two groups.

In another experiment, NOD/SCID mice, (NOD.CB₁₇-Prkdcscid/J) were treated over a time course with 60 mg/kg of MT103 or a sham treatment, and a different lung cancer cell line was used, A549 (an adenocarcinoma), instead of NCI-H226 (a squamous carcinoma). In this second experiment, there was a delayed onset of tumor growth in the treated group, but the subsequent growth curve had a similar slope compared to the control group. Nonetheless, tumors in the treated group were significantly smaller than the control groups (t=30 days).

The examples set forth herein are exemplary and are not intended to limit the scope or spirit of the invention. Many embodiments of the MT103 family have been set forth herein; persons of ordinary skill in these arts will appreciate, after reading this disclosure, additional variations and alternatives that may be accomplished; such variations and alternative would therefore fall within the scope of this disclosure. Patents, patent applications, journal articles, and publications that have been referenced in this application are hereby incorporated by reference herein.

What is claimed is:

1. A method for treating a patient, the method comprising administering to the patient a therapeutically effective amount of a composition comprising a chemical comprising the formula A—Z—Y—X, wherein
A is a bicyclo [2.2.1] heptane group, a heterocyclic group, an alicyclic group, or an aromatic group;
Z is a bond or a linking group;
Y is a group having one of C, S, O, N, or P;
X is —(CH₂)ₙ—X* group, wherein
X* is a H, a halogen, a hydroxyl group, a thiol group, a carboxyl group, an amino group, an alkyl group, an alkoxy group, an alkenyl group, an alkynyl group, a heterocyclic group, or an aromatic group; and
—(CH₂)ₙ is a group where n is an integer between 1 and about 50, inclusive, and one or more of the methylene groups is optionally replaced by O, S, N, C, B, Si, P, C═O, O═S═O, a heterocyclic group, an aromatic group, an NRₐ group, a CR_{b} group, a CR_{c}R_{d} group, or a SiRₑR_{f} where Rₐ, R_{b}, R_{c}, R_{d}, Rₑ, and R_{f} are, each independently, a bond, a pi bond, H, a hydroxyl group, a thiol group, a carboxyl group, a carbamate group, an oxocarbon group, an amino group, an amido group, an amide group, a phosphate group, a sulfonate group, an alkyl group, an alkoxy group, an alkenyl group, an alkynyl group, a heterocyclic group, an aromatic group, or a part of a ring group.

2. The method of claim 1, wherein Y is one of

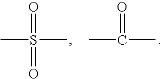

3. The method of claim 1, wherein Y comprises at least one substituent that is H, alkyl group, alkenyl group, alkynyl group, hydroxyl group, or halogen.

4. The method of claim 1, wherein Z is a —(CH₂)ₚ— group, where p is an integer between 1 and 10, inclusive, and one or more of the methylene groups is optionally replaced by O, S, N, C, B, Si, P, C═O, O═S═O, a heterocyclic group, an aromatic group, an NR_{g} group, a CRₕ group, a CRᵢRⱼ group, or a SiRₖR_{I} where R_{g}, Rₕ, Rᵢ, Rⱼ, Rₖ, and Rₗ are, each independently, a bond, H, a halogen, a hydroxyl group, a thiol group, a sulfonate group, a carboxyl group, an amino group, an amido group, an amide group, a phosphate group, an alkyl group, an alkenyl group, an alkynyl group, an alkoxy group, an oxo group, an ether, an ester, a ketone, a carboxyl, a cyclic group, an alicyclic group, an aromatic group, a heterocyclic group, or a part of a ring group.

5. The method of claim 1, wherein at least one of A, X, Y, and Z, further comprises at least one substituent group chosen to be a halogen, H, hydroxyl group; ester group; ether group; an oxo acid group, an oxocarbon group, an oxo carboxylic acid group, an oxo group, a ketone group; nitro group; azido group; sulfhydryl group; alkanoyl group, a carboxamido group; an alkyl group, an alkenyl group, an alkynyl group, an alkoxy group, an aryloxy group; an alkylthio group; an alkylsulfinyl group; an alkylsulfonyl group; an aminoalkyl group, an aralkoxy group, a heteroaromatic group, a heterocyclic group, a heteroalicyclic group, an amine group, an amide group, an amidium ion group, an amine imide group, an amine oxide group, an aminium ion group, an aminonitrenes group, a nitrene group, an aminoxide group, a nitrile group, a nitrile imide group, a sulfonic acid group, a sulfate group, a sulfonate group, a sulfamic acid group, a sulfane group, a sulfatide group, a sulfenamide group, a sulfene group, a sulfenic acids group, a sulfenium ion group, a sulfenyl group, a sulfenylium ion group, a sulfenyl nitrene group, a sulfenyl radical group, a sulfide group, a sulfilimine group, a sulfimide group, a sulfimine group, a sulfinamide group, a sulfinamidine group, a sulfine group, a sulfinic acid group, a sulfinic anhydride group, a sulfinimine group, a sulfinylamine group, a sulfolipid group, a sulfonamide group, a sulfonamidine group, a sulfonediimine group, a sulfone group, a sulfonic acids group, a sulfonic anhydride group, a sulfonamide group, a sulfonium group, a sulfonphthalein group, a sulfonylamine group, a sulfoxide group, a sulfoximide group, a sulfoximine group, a sulfur diimide group, a thiol group, a thioacetals group, a thioaldehyde group, a thioaldehyde S-oxide group, a thioanhydride group, a thiocarboxylic acid group, a thiocyanate group, a thioether group, a thiohemiacetals group, a thioketone group, a thioketone S-oxide group, a thiolates group, a thionylamines group, an alcohol group, a carboxylic group, an aldehydes group, a ketone group, an ether group, an ester group, a phosphane group, a phosphanyliden group, a phosphatidic acid group, a phosphazenes group, a phosphine oxide group, a phosphine group, a phosphinic acid group, a phosphinidenes group, a phosphinous acid group, a phosphoglycerides group, a phospholipid group, a phosphonic acid group, a phosphonitriles group, a phosphonium group, a phosphonium ylide group, a phosphono group, a phosphonous acid group, a phosphoramide group, or a phosphorane group.

6. The method of claim 1, wherein A has the formula A₁-A₇ with at least one substituent chosen from R₁-R₅, and R₁′-R₅′; Z has at least one substituent chosen from R₆ and R₇; Y has at least one substituent chosen from B₁ and B₂, and X has at least one substituent chosen from R₈, R₉, R₉R₉″, so that the chemical formula is 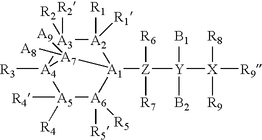
wherein
A₁-A₇ independently comprise C, S, O, or N;
A₈ and A₉ are, each independently, H, an alkyl group, an alkenyl group, an alkynyl group, or a halogen group except that A₈ and A₉ may be combined to form a single group comprising a C or O having a double bond to A₇;
R₉″, B₁, B₂, R₁-R₅, R₁′-R₅′, R₆, and R₇, are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group; and
R₈ an R₉ comprise at least four atoms.

7. The method of claim 6, wherein at least one of R₈ and R₉ comprises a cyclic group.

8. The method of claim 7, wherein the cyclic group is a heterocyclic group.

9. The method of claim 8 wherein the heterocyclic group comprises a ring having at least one member of the group consisting of S, N, O and P.

10. The method of claim 7, wherein the cyclic group is alicyclic.

11. The method of claim 7, wherein the cyclic group is aromatic.

12. The method of claim 7 wherein the cyclic group further comprises a substituent group that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

13. The method of claim 6 wherein at least one of B₁ and B₂ comprises O.

14. The method of claim 6 wherein at least one of R₉″, B₁, B₂, R₁-R₅, R₁′-R₅′, R₆, and R₇ comprises a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

15. The method of claim 14 wherein at least one of A₁-A₉ has a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

16. The method of claim 6 wherein A₁-A₉ are C, X is N, Y is S, Z is C, R₉″ is a lone electron pair or a bond, B₁ and B₂ are each O with a double bond to the Y.

17. The method of claim 16 wherein the chemical comprises a formula chosen from the group consisting of

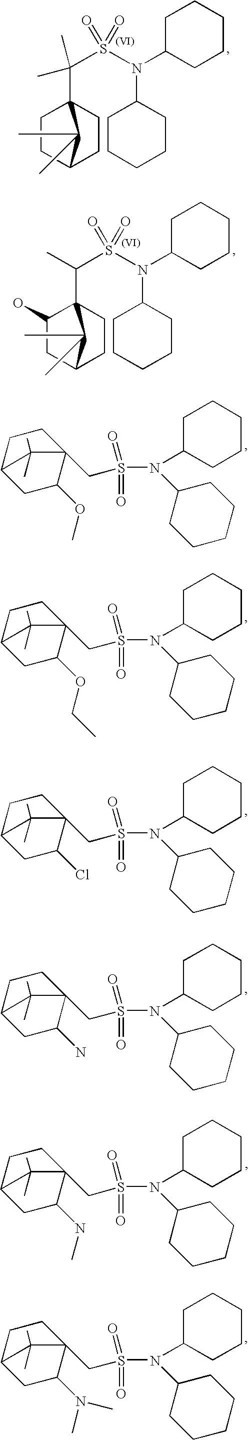

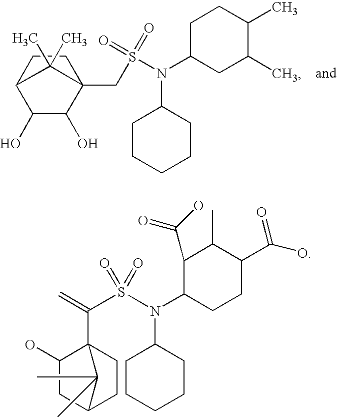

18. The method of claim 6, wherein A₁-A₇ are C; A₈ and A₉ each comprise a methyl group, Z is C, Y is S, B₁ and B₂ are each O with a double bond to the Y, X is N, and R₈ and R₉each comprise a C₆ alicyclic group so that the chemical formula is: 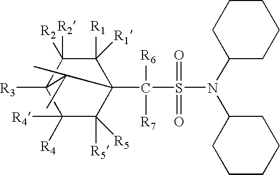
wherein
R₁-R₇ and R₁′-R₅′, are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

19. The method of claim 18 wherein at least one of the cyclic groups depicted in the formula of claim 16 further comprises a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

20. The method of claim 6, wherein A₁-A₇ are C; A₈ and A₉ each comprise a methyl group, Z is C; Y is S, B₁ and B₂ are each O with a double bond to the Y, X is N, and R₈ and R₉each comprise a C₆ aromatic group so that the chemical formula is: 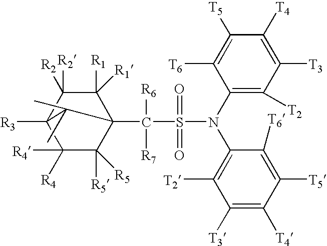
wherein
R₁-R₇, R₁′-R₅′, T₂-T₆, and T₂′-T₆′ are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

21. The method of claim 20 wherein the chemical comprises a formula chosen from the

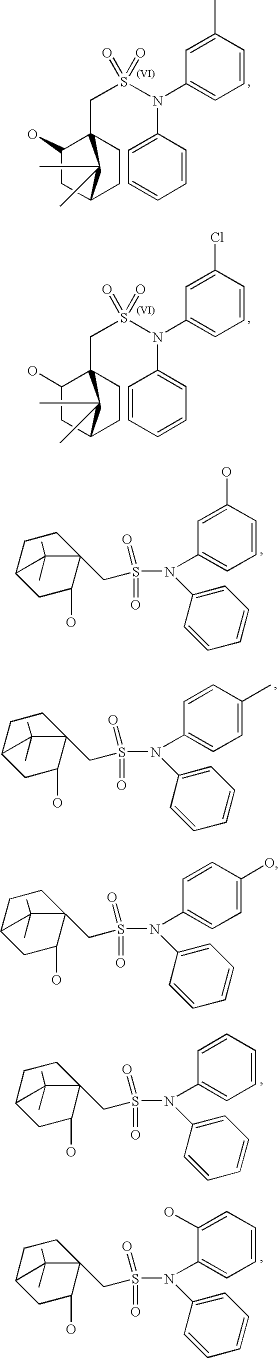

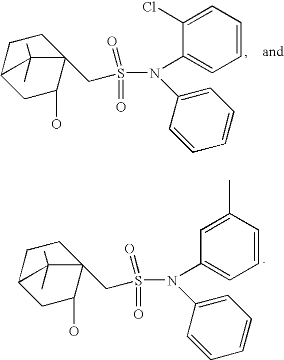

22. The method of claim 6, wherein A₁-A₇ are C; A₈ and A₉ each comprise a methyl group, Z′ is C; Y′ is S, B₁ is O, B₂ is O, X′ is N, and R₉ and R₉ each comprise a methyl group so that the chemical formula is: 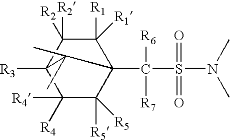
wherein R₁-R₇, and R₁′-R₅′ are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

23. The method of claim 22 wherein at least one of the methyl groups substituent to the N depicted in the formula of claim 22 has at least one substituent independently chosen to be an alkyl group, an alkenyl group, an alkynyl group, a halogen group, a cyclic group, an alicyclic group, an aromatic group, or a heterocyclic group.

25. The method of claim 22 wherein the chemical comprises a formula chosen from the group consisting of

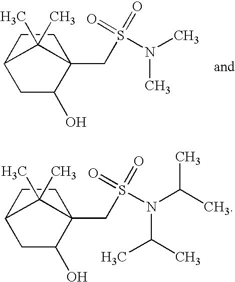

25. The method of claim 6, wherein A₁-A₇ are C; A₈ and A₉ each comprise a methyl group, Z is C; Y is S, B₁ is O, B₂ is O, X is N, R₈ and R₉ each comprise an alicyclic group, and the chemical formula is: 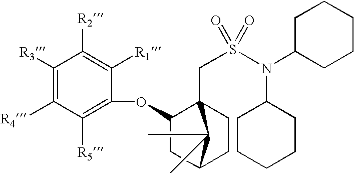
wherein R₁′″-R₅′″, are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group

26. The method of claim 25, wherein at least one of the cyclic groups depicted in the formula of claim 16 further comprises a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

27. The method of claim 25 wherein the chemical comprises a formula chosen from the group consisting of

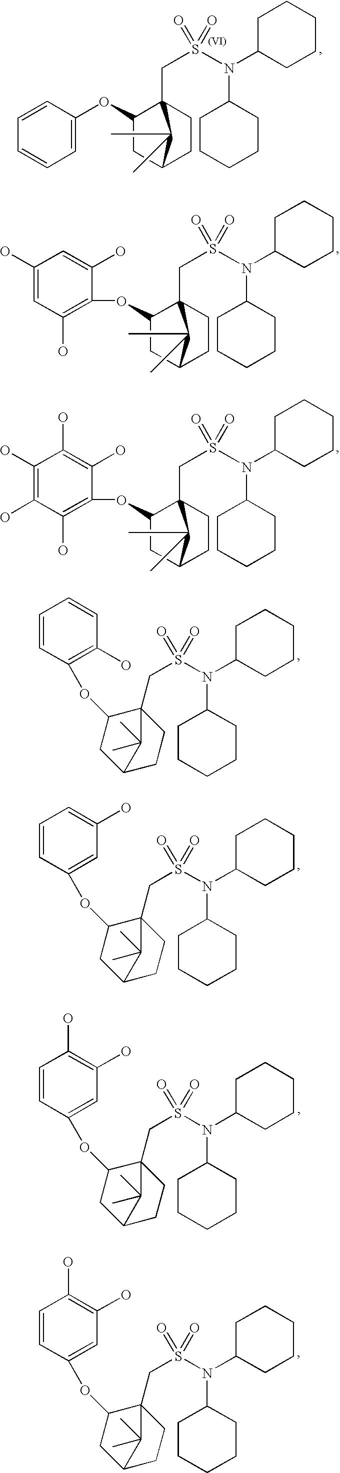

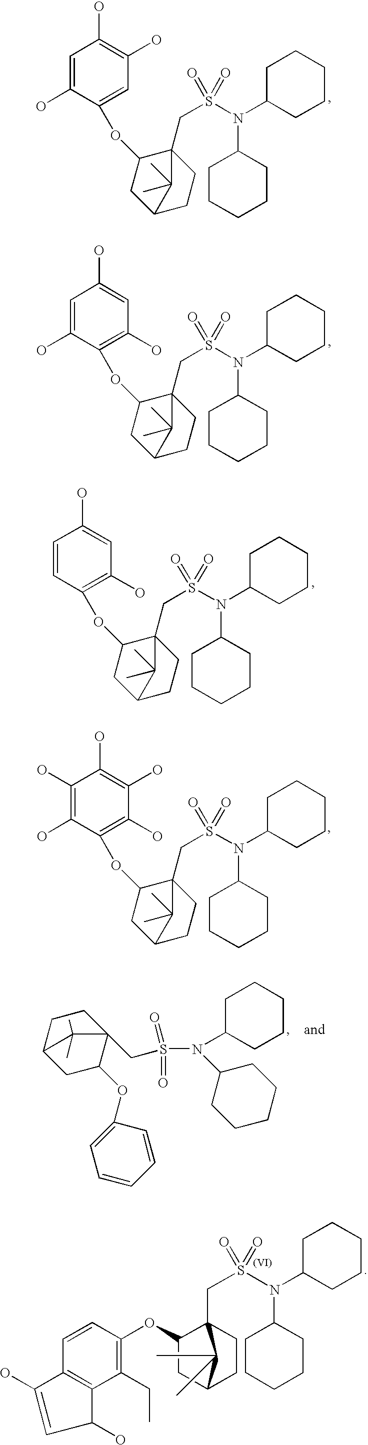

28. The method of claim 4, wherein A has the formula A₁-A₇ with at least one substituent chosen from R₁-R₅, and R₁′-R₅′; Z comprises Z′ and Z₁′ and has at least one substituent chosen from R₆, R₇, R₆′, and R₇′; Y has at least one substituent chosen from B₁ and B₂, and X has at least one substituent chosen from R₈, R₉, and R₉″, so that the chemical formula is 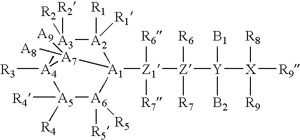
wherein
A₁-A₇ independently comprise C, S, O, or N;
A₈ and A₉ are, each independently, H, an alkyl group, an alkenyl group, an alkynyl group, or a halogen group except that A₈ and A₉ may be combined to form a single group comprising a C or O having a double bond to A₇;
R₉″, B₁, B₂, R₁-R₅, R₁′-R₅′, R₆, R₇, R₆″, and R₇″ are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group; and
R₈ an R₉ comprise at least four atoms.

29. The method of claim 28, wherein at least one of R₈ and R₉ comprises a cyclic group, a heterocyclic group, a heterocyclic group comprising a ring having at least one member of the group consisting of S, N, O and P, an alicyclic group, and an aromatic group.

30. The method of claim 29 wherein the cyclic group further comprises a substituent group that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

31. The method of claim 29 wherein at least one of A₁-A₉ has a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

32. The method of claim 1 wherein A comprises a heterocyclic group comprising formula 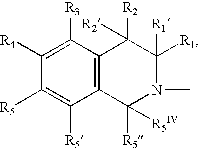
wherein
R₁, R₁′, R₂, R₂′, R₅″, and R₅^{IV} are independently chosen to be a lone electron pair H, a pi bond, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group; and
R₃, R₄, R₅, and R₅′ are independently chosen to be H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

33. The method of claim 32, wherein at least one of R₈ and R₉ comprises a cyclic group, a heterocyclic group, a heterocyclic group comprising a ring having at least one member of the group consisting of S, N, O and P, an alicyclic group, or an aromatic group.

34. The method of claim 32 wherein the formula of claim 30 further comprises a substituent group that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

35. The method of claim 32 wherein at least one of R₁, R₁′, R₂, R₂′, R₅″, and R₅^{IV} has a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

36. The method of claim 32 wherein the chemical comprises a formula chosen from the group consisting of

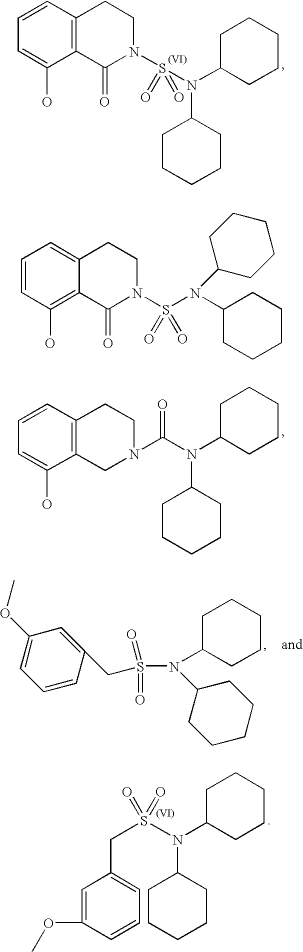

37. The method of claim 4, wherein A has the formula A₁-A₇ with at least one substituent chosen from R₁-R₅, and R₁′-R₅′; Z comprises at least one substituent chosen from R₆, R₇, R₆′, and R₇′; Y comprises at least one substituent chosen from B₁ and B₂, and X is C, and comprises at least one substituent chosen from R₈, R₉, and R₉″, so that the chemical formula is 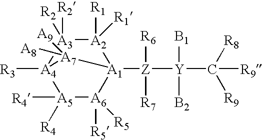
wherein
A₁-A₇ independently comprise C, S, O, or N;
A₈ and A₉ are, each independently, H, an alkyl group, an alkenyl group, an alkynyl group, or a halogen group except that A₈ and A₉ may be combined to form a single group comprising a C or O having a double bond to A₇;
R₉″, B₁, B₂, R₁-R₅, R₁′-R₅′, R₆, R₇, and R₉″ are independently chosen to be a pi bond, a lone electron pair, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group; and
R₈ and R₉ comprise at least four atoms.

38. The method of claim 37, wherein at least one of R₈ and R₉ comprises a cyclic group, a heterocyclic group, a heterocyclic group comprising a ring having at least one member of the group consisting of S, N, O and P, an alicyclic group, or an aromatic group.

39. The method of claim 37 wherein the cyclic group further comprises a substituent group that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

40. The method of claim 37 wherein at least one of A₁-A₉ has a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

41. The method of claim 37 wherein the chemical comprises a formula chosen from the group consisting of

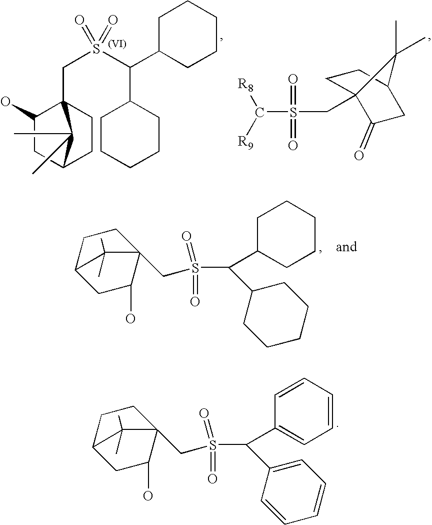

42. A pharmaceutical composition comprising a pharmaceutically acceptable carrier or diluent associated with a therapeutically effective amount of a chemical comprising
a formula of A—Z—Y—X, wherein
A is a bicyclo [2.2.1] heptane group, a heterocyclic group, an alicyclic group, or an aromatic group;
Z is a bond or a linking group;
Y is a group having one of C, S, O, N, or P;
X is —(CH₂)ₙ—X* group, wherein
X* is a H, a halogen, a hydroxyl group, a thiol group, a carboxyl group, an amino group, an alkyl group, an alkoxy group, an alkenyl group, an alkynyl group, a heterocyclic group, or an aromatic group; and
—(CH₂)ₙ is a group where n is an integer between 1 and about 50, inclusive, and one or more of the methylene groups is optionally replaced by O, S, N, C, B, Si, P, C═O, O═S═O, a heterocyclic group, an aromatic group, an NRₐ group, a CR_{b} group, a CR_{c}R_{d} group, or a SiRₑR_{f} where Rₐ, R_{b}, R_{c}, R_{d}, Rₑ, and R_{f} are, each independently, a bond, a pi bond, H, a hydroxyl group, a thiol group, a carboxyl group, a carbamate group, an oxocarbon group, an amino group, an amido group, an amide group, a phosphate group, a sulfonate group, an alkyl group, an alkoxy group, an alkenyl group, an alkynyl group, a heterocyclic group, an aromatic group, or a part of a ring group.

43. The composition of claim 42, wherein the chemical is a salt.

44. The composition of claim 42, wherein the carrier or diluent comprises at least one member of the group consisting of binders, lubricants, disintegrating agents, coloring agents, flavoring agents, flow-inducing agents, and melting agents.

45. The composition of claim 42, wherein at least one of A, X, Y, and Z, further comprises at least one substituent group chosen to be a halogen, H, hydroxyl group; ester group; ether group; an oxo acid group, an oxocarbon group, an oxo carboxylic acid group, an oxo group, a ketone group; nitro group; azido group; sulfhydryl group; alkanoyl group, a carboxamido group; an alkyl group, an alkenyl group, an alkynyl group, an alkoxy group, an aryloxy group; an alkylthio group; an alkylsulfinyl group; an alkylsulfonyl group; an aminoalkyl group, an aralkoxy group, a heteroaromatic group, a heterocyclic group, a heteroalicyclic group, an amine group, an amide group, an amidium ion group, an amine imide group, an amine oxide group, an aminium ion group, an aminonitrenes group, a nitrene group, an aminoxide group, a nitrile group, a nitrile imide group, a sulfonic acid group, a sulfate group, a sulfonate group, a sulfamic acid group, a sulfane group, a sulfatide group, a sulfenamide group, a sulfene group, a sulfenic acids group, a sulfenium ion group, a sulfenyl group, a sulfenylium ion group, a sulfenyl nitrene group, a sulfenyl radical group, a sulfide group, a sulfilimine group, a sulfimide group, a sulfimine group, a sulfinamide group, a sulfinamidine group, a sulfine group, a sulfinic acid group, a sulfinic anhydride group, a sulfinimine group, a sulfinylamine group, a sulfolipid group, a sulfonamide group, a sulfonamidine group, a sulfonediimine group, a sulfone group, a sulfonic acids group, a sulfonic anhydride group, a sulfonamide group, a sulfonium group, a sulfonphthalein group,a sulfonylamine group, a sulfoxide group, a sulfoximide group, a sulfoximine group, a sulfur diimide group, a thiol group, a thioacetals group, a thioaldehyde group, a thioaldehyde S-oxide group, a thioanhydride group, a thiocarboxylic acid group, a thiocyanate group, a thioether group, a thiohemiacetals group, a thioketone group, a thioketone S-oxide group, a thiolates group, a thionylamines group, an alcohol group, a carboxylic group, an aldehydes group, a ketone group, an ether group, an ester group, a phosphane group, a phosphanyliden group, a phosphatidic acid group, a phosphazenes group, a phosphine oxide group, a phosphine group, a phosphinic acid group, a phosphinidenes group, a phosphinous acid group, a phosphoglycerides group, a phospholipid group, a phosphonic acid group, a phosphonitriles group, a phosphonium group, a phosphonium ylide group, a phosphono group, a phosphonous acid group, a phosphoramide group, or a phosphorane group.

46. The composition of claim 42, wherein A has the formula A₁-A₇ with at least one substituent chosen from R₁-R₅, and R₁′-R₅′; Z has at least one substituent chosen from R₆ and R₇; Y has at least one substituent chosen from B₁ and B₂, and X has at least one substituent chosen from R₈, R₉, R₉″, so that the chemical formula is 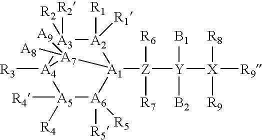
wherein
A₁-A₇ independently comprise C, S, O, or N;
A₈ and A₉ are, each independently, H, an alkyl group, an alkenyl group, an alkynyl group, or a halogen group except that A₈ and A₉ may be combined to form a single group comprising a C or O having a double bond to A₇;
R₉″, B₁, B₂, R₁-R₅, R₁′-R₅′, R₆, and R₇, are independently chosen to be a lone electron pair, a pi bond, H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group; and
R₈ an R₉ comprise at least four atoms.

47. The composition of claim 46, wherein at least one of R₈ and R₉ comprises a cyclic group.

48. The composition of claim 47, wherein the cyclic group is a heterocyclic group.

49. The composition of claim 47, wherein the heterocyclic group comprises a ring having at least one member of the group consisting of S, N, O and P.

50. The composition of claim 47, wherein the cyclic group is alicyclic.

51. The composition of claim 47, wherein the cyclic group is aromatic.

52. The composition of claim 47 wherein the cyclic group further comprises a substituent group that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

53. The composition of claim 46 wherein at least one of B₁ and B₂ comprises O.

54. The composition of claim 46 wherein at least one of R₉″, B₁, B₂, R₁-R₅, R₁′-R₅′, R₆, and R₇ comprises a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

55. The composition of claim 46 wherein at least one of A₁-A₉ has a substituent that comprises H, a halogen, a hydroxyl group, a thiol group, a sulfonate group, a carboxyl group, a carbamate group, an oxocarbon group, an amino group, an amido group, an amide group, a phosphate group, an alkyl group, an alkenyl group, an alkynyl group, an alkoxy group, an oxo group, an ether group, an ester group, a ketone group, a cyclic group, an alicyclic group, an aromatic group, or a heterocyclic group.

56. The composition of claim 46 wherein A₁-A₉ are C, X is N, Y is S, Z is C, R₉″ is a lone electron pair or a bond, B₁ and B₂ are each O with a double bond to the Y.

57. A method of affecting a cell, the method comprising exposing a cell to a chemical comprising
a formula of A—Z—Y—X, wherein
A is a bicyclo [2.2.1] heptane group, a heterocyclic group, an alicyclic group, or an aromatic group;
Z is a bond or a linking group;
Y is a group having one of C, S, O, N, or P;
X is —(CH₂)ₙ—X* group, wherein
X* is a H, a halogen, a hydroxyl group, a thiol group, a carboxyl group, an amino group, an alkyl group, an alkoxy group, an alkenyl group, an alkynyl group, a heterocyclic group, or an aromatic group; and
—(CH₂)ₙ is a group where n is an integer between 1 and about 50, inclusive, and one or more of the methylene groups is optionally replaced by O, S, N, C, B, Si, P, C═O, O═S═O, a heterocyclic group, an aromatic group, an NRₐ group, a CR_{b} group, a CR_{c}R_{d} group, or a SiRₑR_{f} where Rₐ, R_{b}, R_{c}, R_{d}, Rₑ, and R_{f} are, each independently, a bond, a pi bond, H, a hydroxyl group, a thiol group, a carboxyl group, a carbamate group, an oxocarbon group, an amino group, an amido group, an amide group, a phosphate group, a sulfonate group, an alkyl group, an alkoxy group, an alkenyl group, an alkynyl group, a heterocyclic group, an aromatic group, or a part of a ring group.

58. The method of claim 57, wherein the chemical is a salt.

59. The method of claim 57, wherein at least one of A, X, Y, and Z, further comprises at least one substituent group chosen to be a halogen, H, hydroxyl group; ester group; ether group; an oxo acid group, an oxocarbon group, an oxo carboxylic acid group, an oxo group, a ketone group; nitro group; azido group; sulfhydryl group; alkanoyl group, a carboxamido group; an alkyl group, an alkenyl group, an alkynyl group, an alkoxy group, an aryloxy group; an alkylthio group; an alkylsulfinyl group; an alkylsulfonyl group; an aminoalkyl group, an aralkoxy group, a heteroaromatic group, a heterocyclic group, a heteroalicyclic group, an amine group, an amide group, an amidium ion group, an amine imide group, an amine oxide group, an aminium ion group, an aminonitrenes group, a nitrene group, an aminoxide group, a nitrile group, a nitrile imide group, a sulfonic acid group, a sulfate group, a sulfonate group, a sulfamic acid group, a sulfane group, a sulfatide group, a sulfenamide group, a sulfene group, a sulfenic acids group, a sulfenium ion group, a sulfenyl group, a sulfenylium ion group, a sulfenyl nitrene group, a sulfenyl radical group, a sulfide group, a sulfilimine group, a sulfimide group, a sulfimine group, a sulfinamide group, a sulfinamidine group, a sulfine group, a sulfinic acid group, a sulfinic anhydride group, a sulfinimine group, a sulfinylamine group, a sulfolipid group, a sulfonamide group, a sulfonamidine group, a sulfonediimine group, a sulfone group, a sulfonic acids group, a sulfonic anhydride group, a sulfonamide group, a sulfonium group, a sulfonphthalein group,a sulfonylamine group, a sulfoxide group, a sulfoximide group, a sulfoximine group, a sulfur diimide group, a thiol group, a thioacetals group, a thioaldehyde group, a thioaldehyde S-oxide group, a thioanhydride group, a thiocarboxylic acid group, a thiocyanate group, a thioether group, a thiohemiacetals group, a thioketone group, a thioketone S-oxide group, a thiolates group, a thionylamines group, an alcohol group, a carboxylic group, an aldehydes group, a ketone group, an ether group, an ester group, a phosphane group, a phosphanyliden group, a phosphatidic acid group, a phosphazenes group, a phosphine oxide group, a phosphine group, a phosphinic acid group, a phosphinidenes group, a phosphinous acid group, a phosphoglycerides group, a phospholipid group, a phosphonic acid group, a phosphonitriles group, a phosphonium group, a phosphonium ylide group, a phosphono group, a phosphonous acid group, a phosphoramide group, or a phosphorane group.
